(19) United States
(12) Patent Application Publication
(10) Pub. No.: US 2025/0111897 A1
(43) Pub. Date: Apr. 3, 2025

(54) CONCURRENT PROCESSING OF SEQUENCING DATA

(71) Applicant: Illumina, Inc., San Diego, CA (US)

(72) Inventors: James Richard Robertson, San Diego, CA (US); Jason Edward Cosky, San Diego, CA (US); Padmanabhan Ramchandran, San Diego, CA (US); Adam Michael Birnbaum, La Jolla, CA (US); Asaf Moshe Levy, La Jolla, CA (US); Antoine Jean DeJong, Urbana, IL (US); Adam Husar, San Diego, CA (US); Hsu-Lin Tsao, Poway, CA (US)

(73) Assignee: Illumina, Inc., San Diego, CA (US)

(21) Appl. No.: 18/889,691

(22) Filed: Sep. 19, 2024

Related U.S. Application Data
(60) Provisional application No. 63/541,725, filed on Sep. 29, 2023.

(51) Int. Cl. G16B 30/00; G16B 40/20

(52) U.S. Cl. CPC G16B 30/00; G16B 40/20

(57) ABSTRACT

Hardware acceleration may be leveraged for performing secondary analysis. The hardware acceleration may be implemented by utilizing a plurality of field programmable gate arrays (FPGAs) installed on a device. Requests may be made from client processes for performing secondary analysis of sequencing data at a computing device. Each FPGA may be configured with an engine, or set of engines, configured to perform the secondary analysis to service the requests from client process. An FPGA may be configured with a plurality of engines configured for performing secondary analysis. The FPGA may be configured with a single instance comprising different types of engines for performing different types of secondary analysis. The FPGA may be configured with multiple instances of an engine, or set of engines, configured to perform the same or similar type of secondary analysis. The FPGA may share its resources with multiple client processes using one or more shared engines.

### CROSS-REFERENCE TO RELATED APPLICATIONS

This application claims the benefit of U.S. Provisional Patent Application No. 63/541,725, filed Sep. 29, 2023, the disclosure of which is incorporated by reference herein in its entirety.

### BACKGROUND

Next Generation Sequencing (NGS) and variant calling algorithms developed to discover various forms of disease-causing polymorphisms are common tools used to determine the molecular etiology of complex diseases or disorders. Variant discovery from NGS data depends on multiple factors, including reproducible coverage biases of NGS methods and the performance of read alignment and variant calling software. Variant calling platforms provide fast and accurate secondary and tertiary genomic analysis of NGS data for end-to-end implementation of a variant calling pipeline. Variant calling pipelines herein may be implemented in both hardware and software in order to meet the high-speed processing and computational demands of variant calling, particularly in the commercial context.

Field-programmable gate arrays (FPGA) were first deployed as hardware logic for variant calling platforms in single process (SP) environments, in which a single type of secondary analysis would run on a single FPGA installed on a server device at a time. Each FPGA would be reconfigured to perform another form of secondary analysis. However, user workflows on variant caller platforms are often multiplexed, and running applications sequentially for multiple users through single FPGAs would lead to bottlenecking. Moreover, only single version of the software may be installed on the server device at a given time, and so users with specific version requirements would often have to wait their turn for the installation of their desired software versions and corresponding reconfiguration of the FPGA program logic in order to run their applications.

### SUMMARY

Systems, methods, and apparatus are described herein for leveraging hardware acceleration for performing secondary analysis. The hardware acceleration may be implemented by utilizing a plurality of field programmable gate arrays (FPGAs) installed on a device. As described herein, a plurality of requests for hardware acceleration of secondary and/or tertiary analysis of sequencing data may be received at a computing device. The requests may be received from a plurality of client processes operating at one or more client devices. Each FPGA may be configured with an engine, or set of engines, configured to perform the secondary analysis to service the requests from client processes.

Each FPGA may be assigned as a dedicated FPGA for a client process. An FPGA may be configured with a plurality of engines configured for performing secondary analysis. Each engine, or set of engines, may reside in different logical portions of the FPGA. The FPGA may be configured with a single instance comprising different types of engines for performing different types of secondary analysis. For example, the FPGA may be configured with a first engine, or set of engines, for performing mapping/alignment of sequencing data and a second engine, or set of engines, for performing variant calling. As the FPGA may be configured with engines for performing different types of secondary analysis, the sequencing data may be passed downstream, such that each of the engines on the FPGA are concurrently performing different types of secondary analysis for the assigned client process. Each client process being assigned a dedicated FPGA may additionally allow for multiple processes to have their sequencing data processed concurrently.

In another example, an FPGA may share its resources with multiple client processes. For example, the FPGA may be configured with multiple instances of an engine, or set of engines, configured to perform the same or similar type of secondary analysis. Each engine, or set of engines, may reside in different logical portions of the at least one FPGA. Each client process may be assigned to a separate instance of the engine, or set of engines. As such, the same type of secondary analysis may be concurrently performed on the plurality of instances of the engine, or set of engines, on the FPGA.

An FPGA may share its resources with multiple client processes using one or more shared engines. A shared engine may be assigned to multiple client processes for sharing resources on the FPGA. The secondary analysis may be concurrently performed on the shared engine for each client process. For example, the secondary analysis may be performed on the shared engine by time-slicing tasks to be performed on the shared engine for each client process of the plurality of client processes.

### BRIEF DESCRIPTION OF THE DRAWINGS

FIG. 1A illustrates a schematic diagram of a system environment.

FIG. 1B shows an example of one or more secondary analysis subsystems that may be implemented as variant calling platform by the bioinformatics subsystem for performing analysis on sequencing data in response to requests for hardware acceleration of secondary and/or tertiary analysis from one or more clients.

FIG. 1C shows an example system in which the bioinformatics subsystem may leverage one or more portions of a vertical solution stack to service requests for hardware acceleration of secondary and/or tertiary analysis from multiple client subsystems for performing secondary analysis at the bioinformatics subsystem.

FIG. 2A illustrates an example system in which each FPGA of a plurality of FPGAs may be exclusively assigned to client processes.

FIG. 2B illustrates a portion of the system and shows an example configuration of an FPGA for servicing requests for hardware acceleration of secondary and/or tertiary analysis from a dedicated client process.

FIG. 3 is a flowchart depicting an example procedure for performing analysis and processing requests for hardware acceleration of secondary and/or tertiary analysis from client processes.

FIG. 4 is a graph illustrating CPU usage as a percentage over time for performing different types of secondary analysis on FPGAs.

FIG. 5A illustrates an example system in which one or more FPGAs may be shared by client processes.

FIG. 5B illustrates a portion of the system and shows an example configuration of an FPGA for servicing requests for hardware acceleration of secondary and/or tertiary analysis from assigned client processes.

FIG. 5C illustrates a portion of the system and shows another example configuration of an FPGA for servicing requests for hardware acceleration of secondary and/or tertiary analysis from assigned client processes.

FIG. 5D illustrates a portion of the system and shows another example configuration of an FPGA for servicing requests for hardware acceleration of secondary and/or tertiary analysis from assigned client processes using one or more shared engines.

FIG. 5E illustrates a portion of the system and shows an example configuration of the daemon process and a shared engine configured on the FPGA for processing the requests for hardware acceleration of secondary and/or tertiary analysis from multiple assigned client processes.

FIG. 6 is a flowchart depicting another example procedure for performing secondary analysis and processing requests for hardware acceleration of secondary and/or tertiary analysis from client processes.

FIG. 7 is a block diagram illustrating an example computing device.

### DETAILED DESCRIPTION

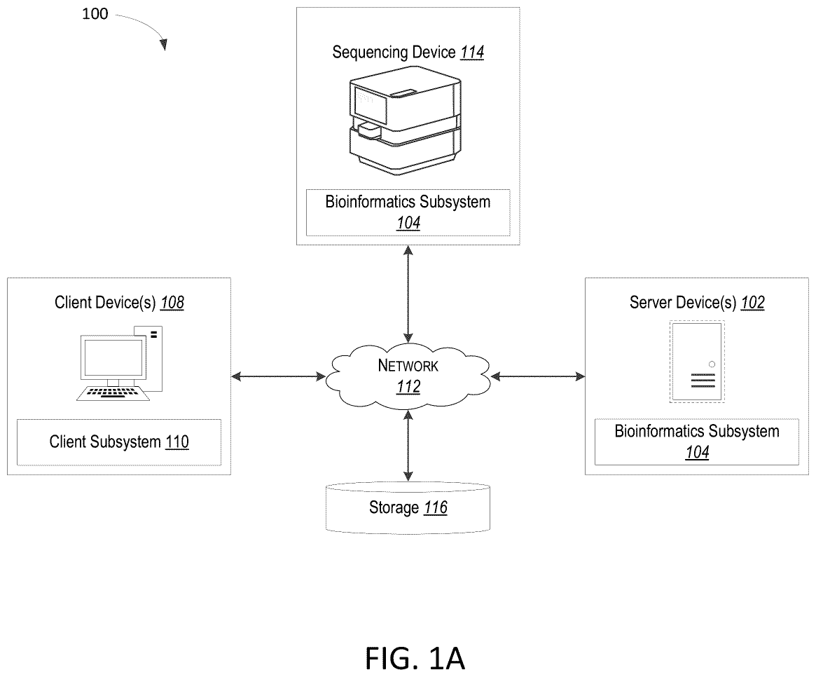

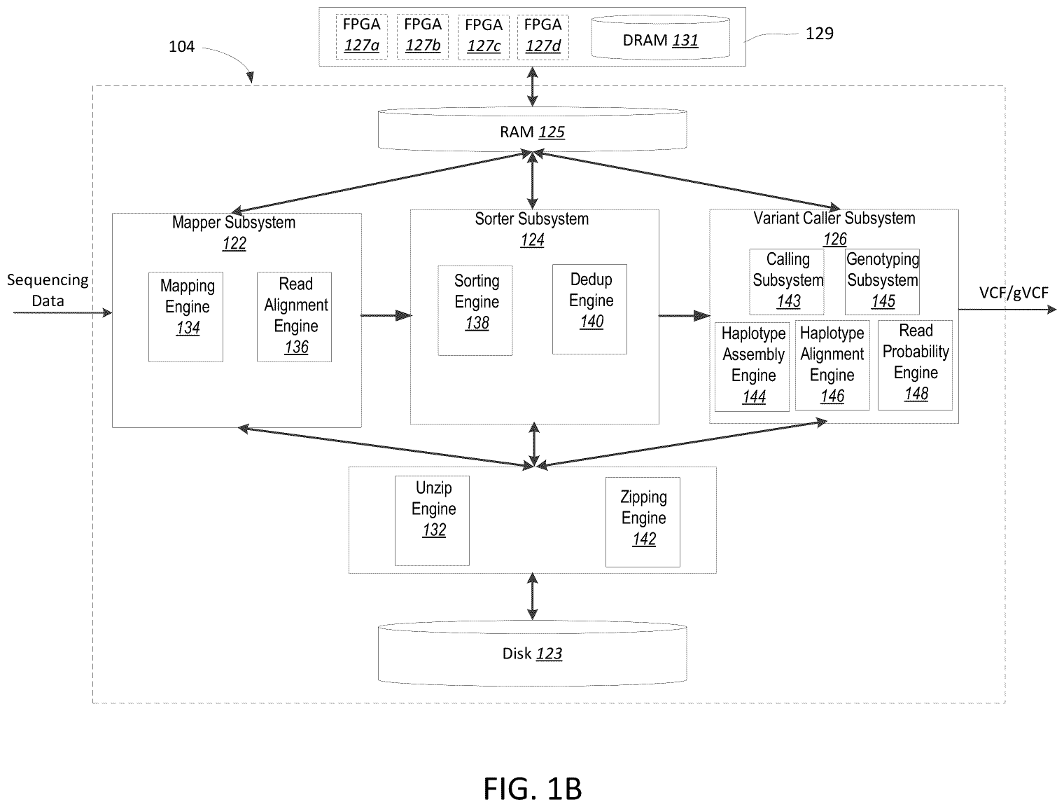

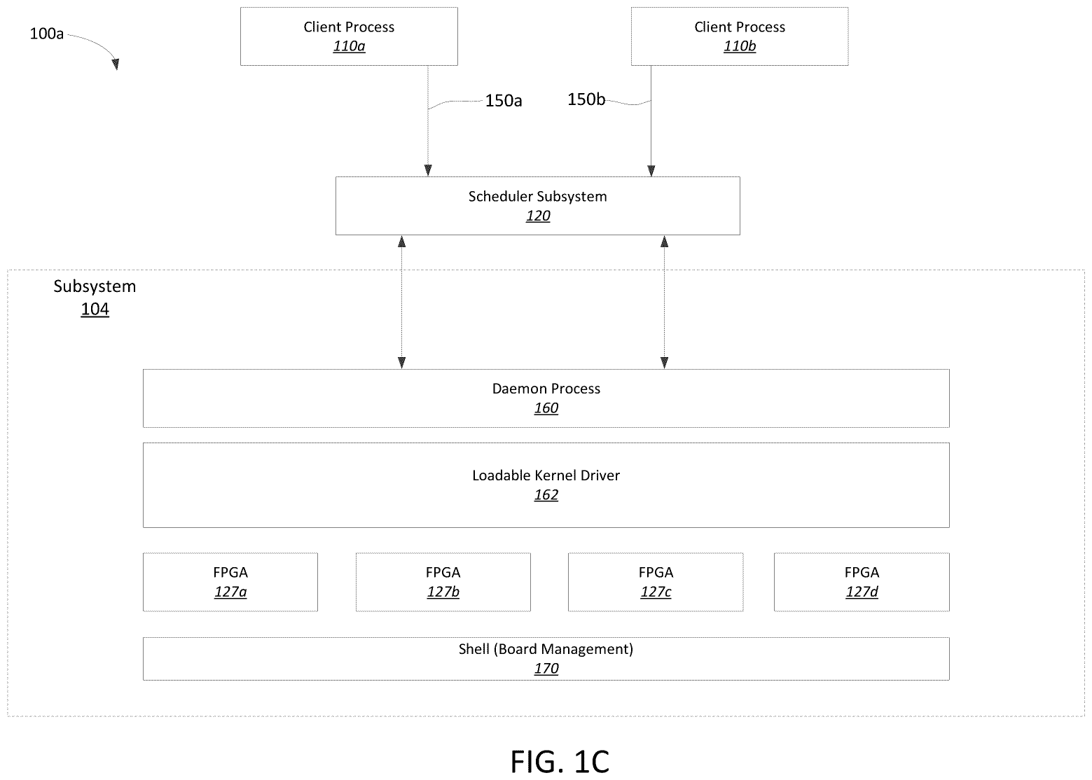

FIG. 1A illustrates a schematic diagram of a system environment (or “environment”) 100, as described herein. As illustrated, the environment 100 includes one or more server device(s) 102 connected to one or more client device(s) 108 and a sequencing device 114 via a network 112.

As shown in FIG. 1A, the server device(s) 102, the client device(s) 108, and the sequencing device 114 may communicate with each other via the network 112 or directly. The network 112 may comprise any suitable network over which computing devices can communicate. The network 112 may include a wired and/or wireless communication network. Example wireless communication networks may be comprised of one or more types of radio frequency (RF) communication signals using one or more wireless communication protocols, such as a cellular communication protocol, a wireless local area network (WLAN) or WIFI communication protocol, and/or another wireless communication protocol. In addition, or in the alternative to communicating across the network 112, the server device(s) 102, the client device(s) 108, and/or the sequencing device 114 may bypass the network 112 and may communicate directly with one another.

As further illustrated in FIG. 1A, the environment 100 may include storage 116. The storage 116 can store information for being accessed by the devices in the environment 100. The server device(s) 102, the client device(s) 108, and/or the sequencing device 114 may communicate with the storage 116 (e.g., directly or via the network 112) to store and/or access information.

As indicated by FIG. 1A, the sequencing device 114 may comprise a device for sequencing a polynucleotide sample. The polynucleotide sample may include human or non-human deoxyribonucleic acid (DNA) or ribonucleic acid (RNA) analytes, including complimentary nucleotide sequences, coding and non-coding sequences, polynucleotide conjugates and analogues, synthetic and recombinant polynucleotides, and amplicons or clones thereof. DNA samples herein may include sample genomic DNA (gDNA) of various ploidy, including gDNA obtained from the diploid genome of humans. Depending on the desired sequencing data and technique employed to obtain the data, the polynucleotide sample may be obtained from single-cell sampling, clonally expanded cell cultures, bulk sampling of tissues; may be obtained from somatic cells, germ line cells, or combinations of individual somatic and germ line cells; and/or may be obtained from healthy or normal cells, diseased or abnormal cells, or cells having (or lacking) a particular phenotypic trait, genomic marker, or mutagenicity. Sequencing data may include data obtained from whole genomic sequencing (WGS), whole exome sequence (WES) data, sequencing a chromosomal region of interest, including coding and non-coding regions, haploid, diploid or polyploid sequencing, single individual (proband) genomic sequencing or proband and corresponding pedigree sequencing, e.g., trios.

The sequencing device 114 may use any number or combination of sequencing techniques. For example, the sequencing device 114 may perform short-read sequencing, including sequencing-by-synthesis (SBS) performed, e.g., on ILLUMINA NOVASEQ sequencers. Short-read sequencing methodologies analyze polynucleotide fragments processed from samples to generate nucleotide reads up to around 600 base pairs in length. Short read methodologies for polynucleotide materials on NGS platforms commonly deploy DNA libraries in which a DNA target (e.g., genomic DNA (gDNA), or complimentary DNA (cDNA)) is processed into fragments and ligated with technology-specific adaptors. NGS workflow using, e.g., an SBS technique, involves loading a DNA library onto a flow cell and hybridizing individual DNA fragments to adapter-specific complimentary oligonucleotides (oligos) covalently bound to a solid support (e.g., flow cell); clustering the individual fragments into thousands of identical DNA template strands (amplicons) through bridge amplification; and, finally, sequencing, in which copy strands are simultaneously synthesized and sequenced on the DNA templates using a reversible terminator-based process that detects signals emitted from fluorophore-labeled single bases as they are added round by round to the copy strands. Because the multiple template strands of each cluster have the same sequence, base pairs incorporated into the corresponding copy strands in each round will be the same, and thus the signal generated from each round will be enhanced proportional to the number of copies of the template strand in the cluster. Various other short-read sequencing implementations herein may include, e.g., real time sequencing; single-molecule sequencing; stochastic sequencing; amplification-free sequencing; sequencing by ligation; pyrosequencing; and/or ion semiconductor sequencing.

As another example, the sequencing device 114 may perform long-read sequencing. While long-read sequencing is performed at much lower throughput compared to short-read sequencers, long-read sequencers can generate reads at kilobase scale. Examples of long read sequencing techniques include Pacific Biosciences' single-molecule real-time (SMRT) sequencing of circular consensus sequences (CCS), and Oxford Nanopore Technologies' nanopore sequencing methodology. In certain embodiments, sequencing data may be generated from a polynucleotide sample using only one sequencing methodology, in which case the environment 100 may include a single sequencing device 114 adapted to perform the particular sequencing methodology, e.g., implementing either a particular short-read or long-read sequencing technique. However, in certain other embodiments, it may be desirable to generate sets of sequencing data from a sample using more than one sequencing methodology, e.g., particular short-read and long-read sequencing techniques, in which case the environment 100 may include either multiple sequencing devices 114 for separately generating short-read and long-read sequencing data of a given sample or a single sequencing device 114 adapted to generate both short-read and long-read sequencing data. For example, ILLUMINA NOVASEQ sequencers may generate long-read sequencing data via tagmentation of long fragment lengths to sample polynucleotide sequences to capture single-molecule, long-read information prior to downstream amplification and processing.

As further indicated by FIG. 1A, the server device(s) 102 may generate, receive, analyze, store, and/or transmit digital data, such as raw sequencing data for determining nucleotide-base calls or sequencing nucleic acid polymers. As shown in FIG. 1A, the sequencing device 114 may generate and send (and the server device(s) 102 may receive) nucleotide reads and/or other sequencing data for being analyzed by the server device(s) 102 for base calling and/or variant calling. The server device(s) 102 may also communicate with the client device(s) 108. In particular, the server device(s) 102 may send data to the client device(s) 108, including sequencing data or other information and the server device(s) 102 may receive input from users via client device(s) 108.

The server device(s) 102 may comprise a distributed collection of servers where the server device(s) 102 include a number of server devices distributed across the network 112 and located in the same or different physical locations. Further, the server device(s) 102 may comprise a content server, an application server, a communication server, a web-hosting server, or another type of server.

As further shown in FIG. 1A, the server device(s) 102 and/or the sequencing device 114 may include a bioinformatics subsystem 104, The bioinformatics subsystem 104 may include software and/or hardware utilized by the server device(s) 102 for processing sequencing requests and/or data, as described herein. The bioinformatics subsystem 104 may be included in a single server device 102 or sequencing device 114, or may be distributed across multiple devices. The bioinformatics subsystem 104 may include a sequencing system that spans multiple layers of software and/or hardware for servicing requests for hardware acceleration of secondary and/or tertiary analysis at the bioinformatics subsystem 104. The sequencing services may include primary analysis for generating sequencing data from images, secondary analysis for determining variant calls or other results from the sequencing data, and/or tertiary analysis for further analyzing the results of the secondary analysis using algorithms to better understand the results, for example.

The bioinformatics subsystem 104 may perform all or a portion of primary data analysis, including, for example, analysis of raw read data (e.g., signal analysis), targeted generation of legible sequencing reads (base calling) and scoring base quality. In addition to performing primary analysis functions, the bioinformatics subsystem 104 may generate data for processing and/or transmitting to other devices for performing secondary and/or tertiary analysis functions. The data may be embodied as one or more files, as described herein.

Each client device 108 may generate, store, receive, and/or send digital data. In particular, the client device 108 may receive sequencing metrics from the sequencing device 114. Furthermore, the client device 108 may communicate with the server device(s) 102 to receive input data (e.g., comprised in one or more files) comprising nucleotide base calls and/or other metrics. The client device 108 may present or display information pertaining to the nucleotide-base call within a graphical user interface to a user associated with the client device 108.

The client device(s) 108 illustrated in FIG. 1A may comprise various types of client devices. In examples, the client device 108 may include non-mobile devices, such as desktop computers or servers, or other types of client devices. In other examples, the client device 108 may include mobile devices, such as laptops, tablets, mobile telephones, or smartphones.

As further illustrated in FIG. 1A, each client device 108 may include a client subsystem 110. The client subsystem 110 may include software and/or hardware utilized by the client device 108 for processing sequencing requests and/or data, as described herein. The client subsystem 110 may span multiple layers of software and/or hardware. The client subsystem 110 may be included in a single client device 108 or may be distributed across multiple client devices 108.

The client subsystem 110 may comprise a sequencing application. The sequencing application may be a web application or a native application stored and executed on the client device 108 (e.g., a mobile application, desktop application). The sequencing application may include instructions that (when executed) cause the client device 108 to receive data from the sequencing device 114 and/or the server device(s) 102 and present, for display at the client device 108, data to the user of the client device 108.

Client processes may be operated on one or more of the client device 108, the server device 102, and/or the sequencing device 114 for requesting hardware acceleration of secondary and/or tertiary analysis from the bioinformatics subsystem 104. For example, client processes executing on any of the client device(s) 108, server devices (102) and/or sequencing device 114 may transmit requests for hardware acceleration of secondary and/or tertiary analysis at the bioinformatics subsystem 104. The bioinformatics subsystem 104 may load and/or execute different bitstreams to perform different types of secondary analysis and/or tertiary analysis to support requests from the client processes.

The secondary analysis described herein may result in variant calls and/or variant call files generated from the sequencing data. Variant calling pipelines herein may include small variant calling different variant classes, including small variant calling for identification of single nucleotide polymorphisms (SNPs) or insertions or deletions (indels) of generally 50 bp or fewer; copy number variant (CNV) calling for detection of large insertions and deletions associated with genomic copy number variation of generally from 50 bp to several mb; short tandem repeat (STR) calling for detection of highly polymorphic microsatellites of recurring DNA motifs of ˜2-6 bp, and structural variant (SV) for detection of large complex variant structures generally above 1000 kb, and may include a variety of variant classes, including large insertions and deletions, including CNVs, multi-allelic CNVs, mobile element insertions (MEI), translocations, inversions, duplications. STR and SV variant classes are believed to have a disproportionate effect on gene expression compared to SNVs and indels. SV and STR variant classes have a disproportionate effect on gene expression compared to SNPs and indels. However, given complexity and variety these variant classes, STR and SV calling generally implement multiple algorithmic approaches and deep whole genome sequencing to accurately identify and genotype variants in these different classes.

FIG. 1B shows an example of one or more secondary analysis subsystems that may be implemented as variant calling platform by the bioinformatics subsystem 104 for performing secondary analysis on sequencing data in response to requests from one or more client devices 108. As shown in FIG. 1B, the bioinformatics subsystem 104 may implement one or more secondary analysis subsystems, such as a mapper subsystem 122, a sorter subsystem 124, and/or a variant caller subsystem 126, to perform secondary analysis on the sequencing data. Though these subsystems are provided as examples, it will be understood that each subsystem may perform tasks that may be combined in a single subsystem or provided in additional subsystems on the bioinformatics subsystem 104. For example, one or more tasks performed by the sorter subsystem 124 may be implemented by the mapper subsystem 122 and/or the variant caller subsystem 126.

Each secondary analysis subsystem may perform a different task or set of tasks. Certain tasks of the secondary analysis subsystem may be agnostic to sequencing methodology and may be performed across sequencing data forms (e.g., short- and/or long-read data forms). Conversely, certain other tasks of the secondary analysis subsystem may be unique to the sequencing data forms. Tasks of the secondary analysis subsystem may be unique or agnostic to particular techniques implementing a variant calling pipeline (e.g., de novo assembly-based or read-alignments based variant calling approaches). Tasks of the secondary analysis subsystem may be unique or agnostic to the variant class being called, with a number of tasks being developed to specifically support haplotype-resolved, de novo variant calling for SVs and STRs, including tasks associated with aligning, phasing, assembling, variant calling, and/or genotype validation and reporting, that may be common to SV and STR calling strategies.

The mapper subsystem 122 may implement an assembly-based VC pipeline in which the mapper subsystem 122 performs non-reference based (de novo) assembly of reads into contigs (e.g., using a De Bruijn graph). The mapper subsystem 122 may also implement a read-based VC pipeline in which the mapper subsystem 122 aligns reads to reference genome.

The mapper subsystem 122 may receive the sequencing data as input data in a predefined file format, such as, but not limited to, a FASTQ file, per-sample, BCL file, or another sequencing data format that is capable of being recognized for processing. A FASTQ file may include a text file that contains the sequence data from clusters that pass filter on a flow cell. The FASTQ format is a text-based format for storing both a biological sequence (e.g., such as a nucleotide sequence) and corresponding quality scores of the biological sequence. In one or more cases, the bioinformatics subsystem 104 may process the sequencing data to determine the sequences of nucleotide bases in DNA and/or RNA segments or oligonucleotides.

The mapper subsystem 122 may utilize one or more engines to perform mapping and/or aligning of the sequencing data. Each engine may implement hardware and/or software for being used as described herein. The mapper subsystem 122 may receive the sequencing data in a compressed FASTQ file or a decompressed FASTQ file. For example, any one or more of the secondary analysis subsystems may include or use an unzip engine 132 to decompress the FASTQ file or other files that are received in a compressed format (e.g., retrieved from compressed format from disk 123). The decompressed sequencing data may include one or more reads for being mapped to and/or aligned with a reference genome. The mapper subsystem 122 may utilize a mapping engine 134 for mapping the reads of the sequencing data to the reference genome. The mapping engine 134 may generate seeds from the sequencing data and look for matches to a reference genome. The seeds may include patterns of aligned portions of the sequencing data that match or fail to match with the reference genome. The mapping engine 134 may iterate by a seed interval to populate a hash table of extracted seeds from the genome to return a hash table of reference genome and compare with sample data to identify a match to the seed. Longer seeds can identify longer matches and reduce alignment time, shorter seeds can produce more matches with longer alignment time.

The results of the mapping engine 134 may be refined by a read alignment engine 136 of the mapper subsystem 122. The read alignment engine 136 may include one or more algorithms configured to align a location of the one or more reads with a location of the reference genome. In an example, the aligning algorithm may include a Smith-Waterman algorithm. The read alignment engine 136 may perform alignment on the locations of each read with a highest density of seed matches or a density above a threshold when compared to other locations of the read. The read alignment engine 136 may compare each position of a read against each candidate position of the reference genome. These comparisons may correspond to a matrix of potential alignments between the read and the reference genome. For each of these candidate alignment positions, the read alignment engine 136 may generate scores that may be used to evaluate whether the best alignment passing through that matrix cell reaches it by a nucleotide match or mismatch (e.g., diagonal movement), a deletion (e.g., horizontal movement), or an insertion (e.g., vertical movement). An insertion or deletion may be referenced as an indel. A match between the read and the reference genome may provide a bonus on the score. A mismatch or indel may impose a penalty. The overall highest scoring path through the matrix may be the alignment that is chosen. The values chosen for scores by the read alignment engine 136 may indicate how to balance, for an alignment with multiple possible interpretations, the possibility of an indel as opposed to one or more SNPs, or the preference for an alignment without clipping. It will be understood that the tasks performed by a given engine, such as the mapping engine 134, may be combined with the tasks performed by another engine, such as the read alignment engine, in a single engine (e.g., mapping engine, map/align engine, etc.).

After the read alignment is performed at the mapper subsystem 122, the mapped/aligned sequencing data may be passed downstream to the sorting subsystem 124 to sort the reads by reference position, and polymerase chain reaction (PCR) or optical duplicates are optionally flagged. The output from the mapper subsystem 122 may be sent directly to the sorter subsystem, or the output may be stored to disk 123 and retrieved from disk by the sorter subsystem 124. Any of the secondary analysis subsystems may use the zipping engine 142 to compress data for being stored to the disk 123 and/or the unzip engine 132 to decompress the data for processing. The zipping engine 142 may compress the sequencing data into a compressed format, such as a compressed file, for storage and/or downstream processing (e.g., variant calling). The compressed file may be a compressed binary alignment/map (BAM) format, a compressed reference-oriented alignment map (CRAM) format, and/or another file format for processing and/or transmitting to other devices. The BAM format may be an alignment format for storing reads aligned to a reference genome. The BAM format may support short and long reads produced by different sequencing devices 114. The BAM format may be a compressed, binary format that is machine-readable. BAM files may show alignments of the reads received in the data received from the sequencing device 114. CRAM files may be stored in a compressed columnar file format for storing biological sequences. The unzip engine 132 and/or the zipping engine 142 may each be implemented in hardware and/or software.

The sorter subsystem 124 may utilize a sorting engine 138 to sort the reads by reference position. A sorting phase may be performed by the sorting engine 138 of the sorter subsystem 124 on aligned reads. A dedup engine 140 may be utilized to flag and/or remove duplicates. The dedup engine 140 may implement a duplicate-marking algorithm. The duplicate-marking algorithm may group aligned reads into subsets in which each of the members of each subset are potential duplicates. For two pairs of subsets to be duplicates that may be identified as having identical alignment coordinates at both ends and/or identical orientations. Additionally, an unpaired read may be marked as a duplicate when it has an identical coordinate and orientation with either end of any other read, whether a paired read or an unpaired read. Unmapped or read pairs may not be marked as duplicates. When the dedup engine 140 identifies a group of duplicates, it may select the best of the group and mark the others with a PCR or optical duplicate flag. For this comparison, duplicates may be scored based on an average sequence Phred quality score. Paired reads may receive the sum of the scores on both ends, while unpaired reads may receive the score of one mapped end. This score may be used to preserve the reads with the highest quality base calls.

The variant caller subsystem 126 may be used to call variants from the aligned and sorted reads in the sequencing data. For example, the variant caller subsystem 126 may receive the mapped/aligned/sorted/deduplicated reads as input and process the reads to generate variant data to be included as output. The output may be in the form of a variant call file (VCF) or a genomic variant call format (gVCF) file. The VCF file may include a text file used in bioinformatics for storing gene sequence variations. The VCF file may indicate the variations in the sequencing data and/or the reference genome. The gVCF may include an extended format, which may include additional information about “blocks” that match the reference and/or quality scores.

The variant caller subsystem 126 may comprise a calling subsystem 143 and/or a genotyping subsystem 145. As the variant caller subsystem 126 receives the sequencing data, the calling subsystem 143 may identify callable regions with sufficient aligned coverage. The callable regions may be identified based on a read depth. The read depth may represent a number of reads that include any base call at a particular reference genomic position. Sometimes the wrong base may be incorporated into a DNA fragment identified in the sequencing data. For example, a camera in the sequencing device 114 may pick up the wrong signal, the mapper subsystem 122 may misplace a read, or a sample may be contaminated to cause an incorrect base to be called in the sequencing data. By sequencing each fragment numerous times to produce multiple reads, there is a confidence or likelihood that identified variants are true variants and not artefacts from the sequencing process. The read depth represents the number of times each individual base has been sequenced or the number of reads in which the individual base appears in the sequencing data. The higher the read depth at a given position, the greater the level of confidence in variant calling at that position.

The callable regions may be the regions that are passed downstream to the genotyping subsystem 145 for calling variants from the callable region. For example, the genotyping subsystem 145 may compare the callable region to a reference genome for variant calling. After the callable region is identified, the calling subsystem 143 may pass the callable region to the genotyping subsystem 145, which may turn the callable region into an active region for generating potential positions in the active region where there may be variants. The active region may identify areas where multiple reads agree or disagree with the reference genome, and windows may be selected around the active regions for processing for variant calling. The genotyping subsystem 145 may identify a probability or call score of whether a potential position includes a variant.

The genotyping subsystem 145 may include and/or implement one or more engines for performing variant calling on one or a combination of variant classes, including small variant (e.g., SNPs and small indels), copy number variants, small tandem repeats, paralogs, fragments, and structural variants (e.g., large insertions and deletions, multi-allelic CNVs, mobile element insertions (MEI), translocations, inversions, duplications). The genotyping subsystem 145 may include a haplotype assembly engine 144. The haplotype assembly engine 144 may be implemented for performing physical and/or genotype phasing for haplotype-resolved variant calling. Phasing may be performed according to various techniques, including, for example, trio binning, computational phasing, and orthogonal phasing, or a combination of these techniques.

The genotyping subsystem 145 may include or implement a haplotype assembly engine 144. In one example, the haplotype assembly engine 144 may include an algorithm that is implemented to assemble overlapping reads in each active region. The haplotype assembly engine 144 may include a graph engine or graph algorithm, as the haplotype assembly engine 144 may assemble overlapping reads in each active region into a graph, such as a De Bruijn graph (DBG), for example. The graph-based method may use alt-aware mapping for population haplotypes that may be stitched into the reference with known alignments to establish alternate graph paths that reads could seed-map and align to. The haplotype assembly engine 144 may reduce mapping ambiguity because reads that contain population variants may be attracted to the specific regions where variants may be observed.

The DBG may be a directed graph based on overlapping K-mers (length K sub-sequences) in each read or multiple reads. When each read is identical, the DBG is linear. Where there are differences, the graph may form bubbles of multiple paths diverging and rejoining. If the local sequence is too repetitive and the length K is too small, cycles may form, which may invalidate the graph. Different values of K may be attempted until a cycle-free graph is obtained. From this cycle-free DBG, each possible path may be extracted to produce a complete list of candidate haplotypes (e.g., hypotheses for what the true DNA sequence may be on at least one strand).

Each candidate haplotype may be aligned for variant calling. The genotyping subsystem 145 may include and/or implement a haplotype alignment engine 146 for alignment of each of the candidate haplotypes. The haplotype alignment engine 146 may include an algorithm, such as a Smith-Waterman algorithm, that is configured to align each extracted candidate haplotype to the reference genome to identify the variants it represents. The haplotype alignment engine 146 may perform sequence alignment by determining similar regions between two strings of nucleic acid sequences or protein sequences. Instead of looking at the entire sequence, the haplotype alignment engine 146 (e.g., implementing the Smith-Waterman algorithm) may compare segments of possible lengths and optimize a similarity measure. While the Smith-Waterman algorithm is provided as an example algorithm for performing alignment of candidate haplotypes for variant calling, other types of algorithms/engines may be similarly implemented.

The genotyping subsystem 145 may include and/or implement a read probability engine 148 to estimate, for each read-haplotype pair, a probability P (r|H) of observing the read during the sequencing process. The read probability engine 148 may use an algorithm or model to calculate the read likelihood by testing each read against each haplotype to estimate a probability of observing the read assuming the haplotype was the true original DNA sampled. The algorithm or model may be, for example, a hidden Markov model (HMM). For example, the read likelihood may be calculated by evaluating a pair HMM, which may account for the various possible ways the haplotype may have been modified by PCR or sequencing errors into the read observed. The HMM evaluation may use a dynamic programming method to calculate the total probability of any series of Markov state transitions arriving at the observed read.

The genotyping subsystem 145 may generate data (e.g., in a file format) for variant calling based on the output from the read probability engine 148. For example, the genotyping subsystem 145 may form possible diploid combinations of variant events from the candidate haplotypes and, for each combination, calculate the conditional probability of observing an entire read pileup. The calculations may use the constituent probabilities of observing each read, given each haplotype from the evaluation by the read probability engine 148. These calculations may be based on alignment scores generated by the haplotype alignment engine 146. These calculations may feed into a formula or algorithm, such as a Bayesian formula, to calculate a likelihood that each genotype is the truth, given the entire read pileup observed. Genotypes with the highest relative likelihood or with a value indicating a likelihood above a threshold may be reported. The probabilities may be indicated in the data (e.g., VCF or gVCF file) generated by the genotyping subsystem.

The bioinformatics subsystem 104 may perform secondary analysis of the sequencing data at the request of one or more client processes executing on the same or different device by operating the mapper subsystem 122, the sorter subsystem 124, the variant caller subsystem 126, and/or one or more portions thereof. The bioinformatics subsystem 104 may leverage hardware acceleration to implement the secondary analysis, or portions thereof, that are provided by the mapper subsystem 122, the sorter subsystem 124, and/or the variant caller subsystem 126. For example, the bioinformatics subsystem 104 may leverage random access memory (RAM) 125 and/or field programmable gate array (FPGA)-board dynamic RAM (DRAM) 131 on an FPGA board 129. The FPGA board 129 may include multiple FPGAs 127a, 127b, 127c, 127d (collectively referred to as FPGAs 127) that may be leveraged for performing one or more portions of the secondary analysis. Though 4 FPGAs 127 are provided as an example, any number of two or more FPGAs may be implemented, as described herein. Each of the FPGAs 127 may be configured with a bitstream image that is loaded from disk 123 to enable operation of portions of the secondary analysis. The bitstream images may be preconfigured with one or more portions of the mapper subsystem 122, the sorter subsystem 124, and/or the variant caller subsystem 126 for enabling secondary analysis and/or tertiary analysis to be performed using the FPGAs 127. Though subsystems and/or engines are described as being implemented in hardware on FPGAs, one or more portions of a subsystem and/or engine may be operated in hardware, software or a combination thereof. Individual subsystems or engines thereof may be operated in hardware, while others may be operated in software. For example, the mapper subsystem 122 and/or the variant caller subsystem 126 may be implemented, at least in part, in hardware using the FPGAs 127, while the sorter subsystem 124 may be implemented in software. Other configurations would be understood. Additionally, although subsystems and/or engines may be described herein as being implemented for performing secondary analysis, subsystems and/or engines may be similarly implemented for performing tertiary analysis based on results of the secondary analysis. For example, hardware acceleration may be similarly implemented on one or more engines and/or subsystems configured to perform a look-up of variants in clinical or phenotype databases, perform variant annotations, perform tumor mutational burden (TMB), or perform other types of tertiary analysis based on the results of the secondary analysis.

The RAM 125 may operate as host RAM on a host computing device, which may be accessible by the FPGAs 127 on the FPGA board 129. The FPGA board 129 may include an FPGA Peripheral Component Interconnect (PCI) or PCI Express (PCIe) board. The FPGA board 129 may include DRAM 131 that may be implemented by the FPGAs 127 to store and/or access data on the FPGA board 129. The FPGAs 127 may access the DRAM 131 and/or RAM 125 directly for configuring the FPGAs 127 with one or more portions of the mapper subsystem 122, the sorter subsystem 124, and/or the variant caller subsystem 126 for enabling secondary analysis and/or tertiary analysis thereon. For example, the bitstream images may be accessed by the FPGAs 127 and the FPGAs 127 may communicate via input/output streams with the DRAM 131 and/or RAM 125. Each FPGA 127a, 127b, 127c, 127d may be programmed via one or more bitstreams loaded directly from RAM 125 and/or DRAM 131. For example, the bitstreams may be loaded to the DRAM 131 on the FPGA board 129 from the RAM 125, or the bitstreams may be loaded directly from RAM 125 (e.g., bypassing DRAM 131). The RAM 125 and/or DRAM 131 may be partitioned between applications and/or hardware. The bitstreams that are loaded into the FPGAs may allow the FPGAs to operate one or more engines/subsystems, or portions thereof, for enabling hardware acceleration for performing secondary and/or tertiary analysis, as described herein.

The bioinformatics subsystem 104 may leverage the FPGAs 127 as part of a vertical solution stack. FIG. 1C shows an example system 100a in which the bioinformatics subsystem 104 may leverage one or more portions of the vertical solution stack to service requests from multiple client processes for performing secondary analysis and/or tertiary analysis at the bioinformatics subsystem 104. As shown in FIG. 1C, requests may be received at the bioinformatics subsystem 104 from client processes 110a, 110b. For example, each of the client processes 110a, 110b may be executed and/or operated by a client subsystem on the same device or a different device as the device on which the bioinformatics subsystem 104 is operating.

The requests from each of the client processes 110a, 110b can be appropriately managed using a scheduler subsystem 120 for enabling access to other services on the bioinformatics subsystem 104. Though additional requests may be received and managed from any number of client processes. The scheduler subsystem 120 may receive a request 150a from the client process 110a and a request 150b from the client process 110b. The client processes 110a, 110b may each communicate with the scheduler subsystem 120 through standard Berkley (BSD) sockets, an address (e.g., IP address and port), or another communication interface that can be accessed via function calls as an endpoint for sending and/or receiving information. The client processes 110a, 110b may be executing on the same or different versions of software. The scheduler subsystem 120 may be a daemon process or other background process executing on one or more server device(s) 102, one or more sequencing devices 114, and/or distributed across server device(s) 102 and sequencing device(s) 114. The scheduler subsystem 120 may be capable of managing the requests 150a, 150b for secondary analysis or tertiary analysis to be performed by the bioinformatics subsystem 104 to allow the bioinformatics subsystem 104 to load and execute the proper bitstream images for supporting each of the requests 150a, 150b prior to being processed.

The scheduler subsystem 120 may be in communication with one or more other software layers of the vertical solution stack for understanding the current state of resources managed by other software layers for processing the requests of the client processes 110a, 110b by other portions of the vertical solution stack. For example, the scheduler subsystem 120 may be in communication with a daemon process 160 executing on the bioinformatics subsystem 104. The daemon process 160 may be a background process executing on one or more server device(s) 102. The daemon process 160 may manage hardware on the one or more server device(s) 102 in response to requests from client processes. The daemon process 160 may be a child service of the scheduler subsystem 120 that is launched by the scheduler subsystem 120. Scheduler subsystem 120 may launch and monitor its child processes for the duration of its run.

The daemon process 160 may perform several functions for managing access to hardware resources and servicing requests from various client processes. For example, the daemon process 160 may perform board management processes for access to and reconfiguration of hardware resources based on client requests. The daemon process 160 may manage the assignment of clients and/or client requests to given hardware resources for enabling processing of the client requests. The daemon process 160 may perform connection management processes for establishing connection for a given client or client request to hardware resources for services the requests from the client. The daemon process 160 may perform session management processes for establishing a session for one or more connections to one or more engines for servicing client requests to hardware resources for services the requests from the client. The daemon process 160 may perform transmit/receive (TX/RX) queue management processes for managing requests for hardware acceleration of secondary and/or tertiary analysis from various client processes and returning the responses to the requests to the appropriate client processes. The daemon process 160 may perform buffer management processes for managing data stored in the buffers, such as sequencing data for being processed according to the requests and/or buffering the results of the secondary analysis.

The bioinformatics subsystem 104 may include a loadable kernel driver 162, which may be an application resident in memory with which the daemon process 160 may be in communication for facilitating interactions with one or more portions of hardware. For example, the loadable kernel driver 162 may be in communication with the daemon process 160 and/or one or more portions of programmable hardware for servicing the requests for hardware acceleration of secondary and/or tertiary analysis. The loadable kernel driver 162 may be an application resident in memory for facilitating such communication.

The hardware layers of the vertical solution stack at the bioinformatics subsystem 104 may include the field programmable gate arrays (FPGAs) 127 and/or a shell 170. The shell 170 may be a hardware layer that includes lower-level code for controlling hardware functionality on the server device(s) 102. The FPGAs 127 may include more advanced code, such as the partially reconfigurable bitstreams.

The loadable kernel driver 162 may support multiple FPGAs 127. For example, the bioinformatics subsystem 104 may support two FPGAs, four FPGAs, or any number of FPGAs configured to operate as described herein. Each FPGA 127 may comprise a partial reconfiguration bitstream capable of configuring the shell 170 with a base image to enable the FPGA 127 to operate as described herein. The partial reconfiguration bitstream may be plugged in as the base image to the shell 170. The loadable kernel driver 162 may support the FPGAs 127 over Peripheral Component Interconnect Express (PCIe) or another type of serial expansion bus for connecting to one or more peripheral devices. The FPGAs 127 may each be partially reconfigured using a partial bitstream to change the structure of a portion of an FPGA design for performing different forms of secondary analysis and/or on behalf of one or more client processes 110a, 110b.

As shown in FIG. 1C, the daemon process 160 may receive requests 150a, 150b from client processes 110a, 110b and communicate with the loadable kernel driver 162 for updating the partially reconfiguration bitstreams of each of the FPGAs 127 for performing secondary analysis or tertiary analysis in response to the requests. The requests 150a, 150b may be authorized by the scheduler subsystem 120 when the scheduler subsystem 120 determines from the daemon process 160 that an FPGA 127 has been configured with one or more engines for processing the respective request 150a, 150b. The requests 150a, 150b may each include an identifier of a specific engine to be leveraged for performing secondary analysis. For example, with reference to FIG. 1B, the requests 150a, 150b may each include an identifier of the unzip engine 132, the mapping engine 134, and/or the read alignment engine 136 of mapper subsystem 122. The requests 150a, 150b may each include an identifier of the sorting engine 138, the dedup engine 140, and/or the zipping engine 142 of sorter subsystem 124. The requests 150a, 150b may each include an identifier of the haplotype assembly engine 144, the haplotype alignment engine 146, and/or the read probability engine 148 of the variant caller subsystem 126. Each request may include an identifier of a version of the engine to be leveraged for performing the secondary analysis. For example, different versions of an engine having the same engine type may be separately implemented to fulfill requests from client processes operating different versions of software. Each request may include an identifier of the client process from which the request is received, such that the results of the secondary analysis may be provided to the requesting client process.

Referring again to FIG. 1C, the scheduler subsystem 120 may be in communication with the daemon process 160 for identifying the status of one or more FPGAs 127 to authorize requests from various client processes for accessing the one or more FPGAs. The daemon process 160 may monitor the status of each of the FPGAs 127 and communicate the status to the scheduler subsystem 120. When resources are available on one or more of the FPGAs 127, the scheduler subsystem 120 may coordinate the configuration of each available FPGA based on the requests from the client processes 110a, 110b. The scheduler subsystem 120 may instruct the daemon process to configure an available FPGA for performing one or more types of secondary analysis and/or tertiary analysis based on the requests from the client processes 110a, 110b. The daemon process 160 may determine a bitstream image to be uploaded to the one or more FPGAs 127 for enabling the secondary analysis and/or tertiary analysis to service the requests.

In one example, the client processes 110a, 110b may each transmit a respective request 150a, 150b to the scheduler subsystem 120. Each request 150a, 150b may identify one or more engines being requested for performing hardware acceleration of secondary and/or tertiary analysis. Each request 150a, 150b may also identify a version of software being implemented at the respective client process 110a, 110b or a version of the engine being requested. The requests 150a, 150b may be queued until an FPGA 127 that is configured with the requested engine is available for servicing the respective requests 150a, 150b. The daemon process 160 may notify the scheduler subsystem 120 when one of the FPGAs 127 (e.g., FPGA 127a) is configured with the requested engine for performing secondary analysis.

After the scheduler subsystem 120 determines that one of the FPGAs 127 (e.g., FPGA 127a) is configured with the engine, or set of engines, identified in the request 150a for performing secondary analysis, the scheduler subsystem 120 may send the request to the bioinformatics subsystem 104 and/or the daemon process 160. The daemon process 160 may receive the request 150a and may establish a connection with the client process 110a and/or one or more engines, register the client process 110a in memory, and begin servicing the request 150a by communicating with other processes, drivers, and/or layers of the vertical solution stack in the bioinformatics subsystem 104. The connection may be established through standard Berkley (BSD) sockets or another communication interface, an address (e.g., IP address and port), or another communication interface that can be accessed via function calls as an endpoint for sending and/or receiving information.

The scheduler subsystem 120 may similarly receive the request 150b from the client process 110b and after the scheduler subsystem 120 determines that one of the FPGAs 127 (e.g., FPGA 127a or FPGA 127b) is configured with the engine, or set of engines, identified in the request 150b for performing secondary analysis, the scheduler subsystem 120 may send the request to the bioinformatics subsystem 104 and/or the daemon process 160. The daemon process 160 may establish a connection with the client process 110b and/or one or more engines, register the client process 110b in memory, and begin servicing the request 150b by communicating with other processes, drivers, and/or layers of the vertical solution stack in the bioinformatics subsystem 104. The connection may be established through standard Berkley (BSD) sockets or another communication interface, an address (e.g., IP address and port), or another communication interface that can be accessed via function calls as an endpoint for sending and/or receiving information.

The requests 150a, 150b may be received at the daemon process 160 independently or with one or more additional requests from other client processes. The requests 150a, 150b may identify the same or different engine for performing secondary analysis. For example, the requests 150a, 150b may be an initial request from each of the client processes 110a, 110b to perform secondary analysis, which may be a request for an unzip engine 132 and/or a mapping engine 134 to perform mapping of sequencing data. In another example, the request 150a from the client process 110a may be a request for another engine, such as a request for an engine within the variant caller subsystem for variant calling (e.g., the haplotype assembly engine 144, the haplotype alignment engine 146, and/or the read probability engine 148), while the request 150b may be for an engine within the mapper subsystem (e.g., the unzip engine 132 and/or the mapping engine 134).

As illustrated from the examples provided herein, the daemon process 160 may receive multiple requests from multiple client processes operating on various client devices. The daemon process 160 may manage the requests by configuring each of the FPGAs 127 to service the requests for performing secondary analysis. The daemon process 160 may communicate with the loadable kernel driver 162 to service the requests from each of the client processes. For example, the loadable kernel driver 162 may have each of the FPGAs 127 under its control. The loadable kernel driver 162 may support the FPGAs 127 over PCI or PCIe. The daemon process 160 may cause the loadable kernel driver 162 to load one or more engines, via a bitstream, into each FPGA for processing the requests.

As the daemon process 160 may have access to multiple FPGAs 127, there may exist an M-to-N relationship between the number of client processes making requests and FPGA boards. There may be a number of ways each FPGA 127 may be configured for servicing the requests from multiple client processes. There may also be a number of ways to allow the client processes to access each FPGA 127 for processing the requests.

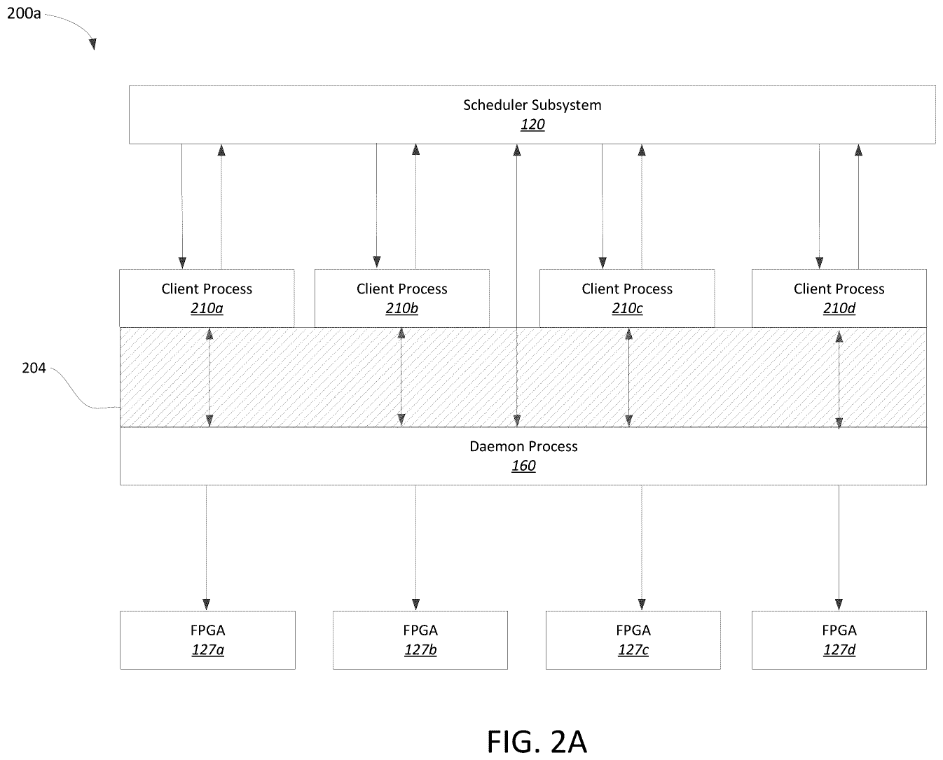

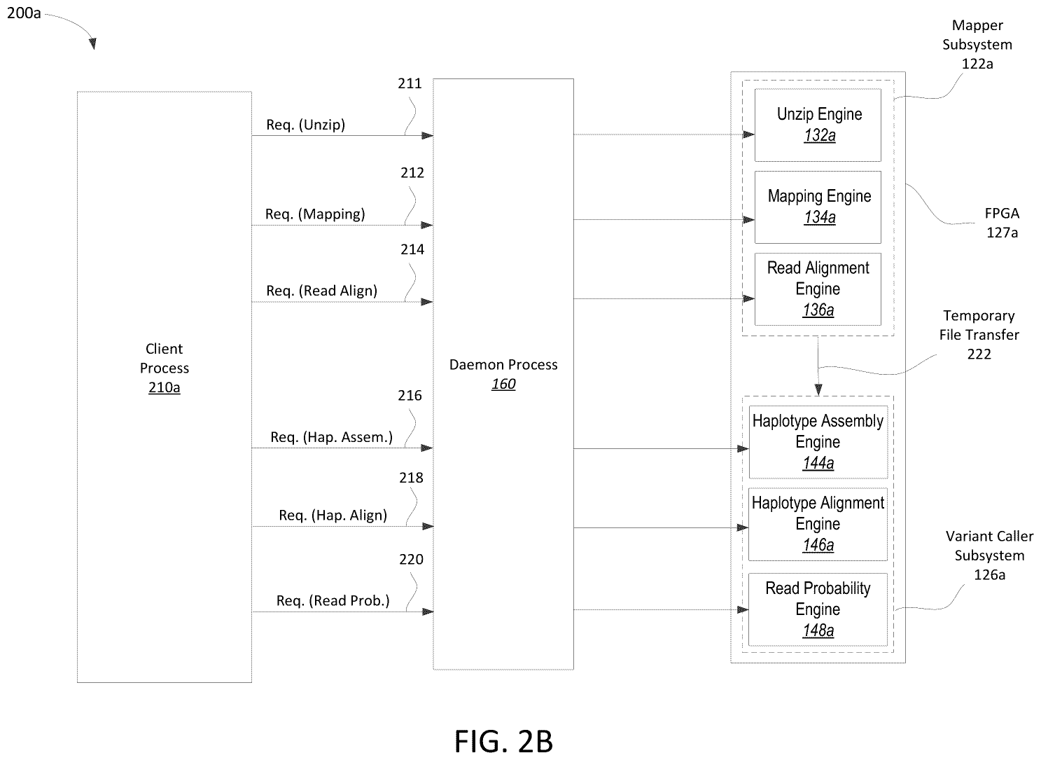

FIG. 2A illustrates an example system 200a in which each FPGA 127 may be exclusively assigned to a client process at a given time. For example, as shown in FIG. 2A, each client process 210a, 210b, 210c, 210d (collectively referred to as client processes 210) may transmit a request to the scheduler subsystem 120 for accessing one or more engines on one of the FPGAs 127 for performing secondary analysis of sequencing data. As each of the client processes 210 may have independent access to a dedicated FPGA for performing secondary analysis, the secondary analysis may be performed for the client processes 210 in an orthogonal mode. In the orthogonal mode, the secondary analysis may be performed for each of the client processes 210 in parallel. The FPGA resources for each of the FPGAs 127 may be utilized to allow each of the client processes 210 to be given dedicated resources.

The scheduler subsystem 120 may be in communication with the daemon process 160. When each of the FPGAs 127 are available, the daemon process 160 may communicate the availability of one or more FPGAs 127 to service the requests to the scheduler subsystem 120. The availability of the resources on the one or more FPGAs 127 may be communicated to the scheduler subsystem via a direct socket with the daemon process 160 that allows the scheduler subsystem 120 to query the daemon process 160 on a communication protocol. The scheduler subsystem 120 may instruct the daemon process 160 to configure the FPGAs 127 based on the requests from the client processes 210. Each of the requests from the client processes 210 may be sent to the daemon process 160 for performing secondary analysis.

In the orthogonal mode, the scheduler subsystem 120 may schedule the same number of client processes 210 for secondary analysis as there are number of FPGAs 127. As each of the client processes 210 may be assigned to a dedicated FPGA, the requests from a client process may be prevented from being sent to other FPGAs. For example, after the client process 210a is assigned to the FPGA 127a and the client process 210b is assigned to the FPGA 127b, the requests from the client process 210a may be directed to the FPGA 127a and may be prevented from being sent to the FPGAs 127b-127d. Similarly, the requests from the other client processes 210b-210d may be prevented from being sent to the FPGA 127a. The daemon process 160 may receive the requests from each of the client processes 210 and leverage the resources of the assigned FPGA.

In response to each of the client processes 210 transmitting a request to the daemon process 160, the daemon process 160 may establish a separate connection with each of the client processes 210 at 204. Upon receipt of the initial request from the client processes 210, the daemon process 160 may establish a session during which each of the connections may be established with the client processes 210. The connections may be established at 204 to allow each of the client processes 210 to read and/or write data to a respective connection. Each connection for a process may be assigned an amount and/or location of memory for processing sequencing data in response to the requests. As a part of the connections that are established at 204, the daemon process 160 may establish an individual stream for sending data to and/or receiving data from the dedicated FPGAs 127.

The daemon process 160 may assign each of the client processes 210 to a dedicated FPGA for servicing request for hardware acceleration of secondary and/or tertiary analysis. For example, the client process 210a may be assigned to FPGA 127a. The client process 210b may be assigned to FPGA 127b. The client process 210c may be assigned to FPGA 127c. The client process 210d may be assigned to FPGA 127d. Though four client processes are shown as being assigned to FPGAs, the number of client processes that are concurrently assigned to FPGAs may vary based on the number of FPGAs. As the number of client processes that are given access to the daemon process 160 and/or the FPGAs 127 in response to a request may be limited by the number of FPGAs 127, requests from additional client processes may be queued at the scheduler subsystem 120 until resources become available at one or more of the FPGAs.

The daemon process 160 may configure each of the FPGAs 127 for servicing the requests from the assigned client process. For example, the daemon process 160 may cause the loadable kernel driver 162 to load one or more engines, via a partial bitstream image, into each FPGA for processing the requests from a dedicated client process. Each FPGA 127 may comprise a partial reconfiguration bitstream that is capable of enabling the FPGA to service requests for hardware acceleration of secondary and/or tertiary analysis from the dedicated client process. In one example, each of the FPGAs 127 may be imaged with a bitstream configured with one or more engines for performing secondary analysis. Each of the FPGAs 127 may be configured and/or reconfigured with a single instance comprising different engines for performing different portions of the secondary analysis as the request from the dedicated client process is completed. This may allow for each of the client processes 210 to have different types of secondary analysis performed in parallel on a single FPGA. Each of the client processes 210 may also perform independent secondary analysis in parallel on separate FPGAs 127. The independent assignment of client processes to dedicated FPGAs may also allow for different FPGAs to be configured for supporting different versions of client processes and/or subsystems. For example, multiple FPGAs may be configured for performing similar types of secondary analysis, but with different versions of engines for supporting different versions of client processes and/or client subsystems.

FIG. 2B illustrates a portion of the system 200a and shows an example configuration of an FPGA 127a (e.g., FPGA 127a shown in FIG. 2A) for servicing requests for hardware acceleration of secondary and/or tertiary analysis from a dedicated client process 210a. Other FPGAs may be similarly configured or differently configured for servicing requests for hardware acceleration of secondary and/or tertiary analysis from other client processes. As shown in FIG. 2B, the client process 210a may be assigned to the FPGA 127a by the daemon process 160. The client process 210a may send one or more requests to the daemon process 160 for requesting one or more types of secondary analysis to be performed on sequencing data.

The sequencing data to be processed may be identified in the request or in a separate request, such as as a sample sheet that is passed to the FPGA 127a for processing. Each flow cell of sequencing data may be fed to the assigned FPGA 127a for processing. The scheduler subsystem 120 may identify how many samples are to be processed in the sequencing data for a given flow cell and may split them up into similar commands or request to be processed on the assigned FPGA 127a. The scheduler subsystem 120 may transmit a command to put the daemon process 160 into a mode to support the commands or requests for processing the sequencing data. The client processes 210 may each read the sequencing data from disk, and at various times may send it through packets to the assigned engines on an FPGA.

The FPGA 127a may be configured with engines for performing different types of secondary analysis. For example, the FPGA 127a may include one or more engines of a mapper subsystem 122a configured to map/align the reads in the sequencing data and one or more engines of a variant caller subsystem 126a configured to call variants from the aligned reads in the sequencing data. As shown in FIG. 2B, the FPGA 127a may be configured with one or more of an unzip engine 132a, a mapping engine 134a, and/or a read alignment engine 136a of the mapper subsystem 122a. Though it will be understood that more or less engines may be configured to perform the mapping and/or alignment of the sequencing data, as described herein. As shown in FIG. 2B, the FPGA 127a may be configured with one or more of a haplotype assembly engine 144a, a haplotype alignment engine 146a, and/or a read probability engine 148a of the variant caller subsystem 126a. Though it will be understood that more or less engines may be configured to perform the variant calling of the sequencing data, as described herein. Similarly, though not shown in FIG. 2B, the FPGA 127a may be configured with one or more additional engines for sorting (e.g., sorting engine), deduplicating (e.g., dedup engine), and/or compressing (e.g., zipping engine) sequencing data. However, the sorting and/or deduplication may be performed by one or more of the engines already configured on the FPGA 127a, or may be performed in software, as described herein.

The engines that are configured on the FPGA 127a may be configured via a bitstream image stored on disk 123 and loaded onto the FPGA 127a via RAM 125. The bitstream image may be preconfigured with a predefined number of engines and/or engine types for performing secondary analysis. For example, the bitstream may be preconfigured with one or more engines of the mapper subsystem 122a configured to map/align the reads in the sequencing data and one or more engines of the variant caller subsystem 126a configured to call variants from the aligned reads in the sequencing data. The bitstream being preconfigured with multiple engines (e.g., engines of the mapper subsystem 122a and engines of the variant caller subsystem 126a) may prevent the FPGA 127a from having to be reconfigured for performing different types of secondary analysis.

As shown in FIG. 2B, the FPGA 127a may be configured with a set or collection of engines in a single instance operating on hardware resources. Each engine, or set of engines, may be configured using different logical portions of the FPGA 127a to perform different types of secondary analysis. The FPGA 127a may include an array of programmable logic blocks and a hierarchy of reconfigurable interconnects that may be programmed by the bitstream images. Each engine, or set of engines, may be configured using a different cluster of one or more engines on the FPGA 127a to support different types of secondary analysis. In the example shown in FIG. 2B, the FPGA 127a may be configured with different logical portions and/or clusters of engines, or a set of the engines, of the mapper subsystem 122a and the engines, or a set of the engines, of the variant caller subsystem 126a. A cluster may comprise a set of engines (e.g., each of the same type or a different type), which may be accessed by the client via a single stream.

When the FPGA 127a is configured with an engine, or set of engines, for performing different types of secondary analysis, a temporary file and/or stream of records in an intermediate/internal format may be generated for passing data between the engine, or sets of engines. For example, the set of engines in the mapper subsystem 122a may generate a stream of records in the intermediate/internal format that is stored in memory for being processed and passed to another engine. If the amount of data in the stream of records occupies a threshold amount of memory, additional data may be spilled to disk and stored as temporary files. If the stream of records can be maintained for less than the threshold (e.g., 20 Gigabytes (GB) of RAM), the temporary file may not need to be generated. The stream of records and/or the temporary file may be transferred at 222 to the set of engines in the variant caller subsystem 126a. The temporary file and/or stream of records may include data from upstream engines for being processed by downstream engines. For example, the temporary file and/or stream of records may include the mapped/aligned reads in a format that may be accepted by the engine or set of engines of the variant caller subsystem 126a. When different engines are included in an FPGA, the temporary file and/or stream of records may include the output of the engine, or set of engines, leveraged for performing another type of secondary analysis. When tertiary analysis is performed via one or more engines on the FPGA, the temporary file and/or stream of records may include the output of the upstream engine(s) (e.g., engines for performing one or more types of secondary analysis). The temporary file and/or stream of records may allow the FPGA 127a to continue performing analysis on the sequencing data for which other types of secondary analysis has been performed without having to generate a separate file, such as a BAM file, a CRAM file, or a Concise Idiosyncratic Gapped Alignment Report (CIGAR) file, for being stored in another location (e.g., on disk 123) for being reloaded for performing subsequent secondary analysis. The temporary file and/or stream of records may use less memory bandwidth (e.g., one or more bits less) and prevent the use of the zipper engine and/or the unzip engine to compress/decompress files, such as BAM files, CRAM files, or CIGAR files.

The configuration of the FPGA 127a may allow for the concurrent performance of different forms of secondary analysis in parallel on the same FPGA 127a. For example, as different logical portions may be configured with engines, or sets of engines, for performing different types of secondary analysis, the same FPGA 127a may be implemented to perform different types of secondary analysis on different portions of the sequencing data in parallel. For example, the client process 210a may send a request 211, 212, 214 for accessing each engine or set of engines 132a, 134a, 136a of the mapper subsystem 122a for mapping/aligning a portion of sequencing data with a reference genome. Each request 211, 212, 214 may identify a corresponding engine 132a, 134a, 136a for performing the requested tasks. However, a single request may be sent to identify a set of engines for performing a corresponding type of secondary analysis. After the mapping/aligning has completed for the portion of the sequencing data, the mapped/aligned reads may be transferred (e.g., via the stream of records and/or the temporary file) to other logical portions of the FPGA 127a for performing subsequent secondary analysis. For example, the client process 210a may send subsequent requests 216, 218, 220 for accessing each engine or set of engines 144a, 146a, 148a of the variant caller subsystem 126a configured to call variants from the aligned reads in the sequencing data. Each request 216, 218, 220 may identify a corresponding engine 144a, 146a, 148a for performing the requested function. While an engine or a set of engines 144a, 146a, 148a are being used to perform a type of secondary analysis (e.g., variant calling) on a portion of the sequencing data at the FPGA 127a, the client process 210a may transmit one or more requests for accessing an engine or a set of engines 132a, 134a, 136a that have been freed up for performing another type of secondary analysis (e.g., mapping/aligning) on another portion of the sequencing data.

Referring again to FIG. 2A, though an example is provided for the configuration of the FPGA 127a, other FPGAs (e.g., FPGAs 127b-127d) may be similarly configured for processing requests from other client processes (e.g., corresponding assigned client processes 210b-210d). Additionally, it will be understood that other arrangements of engines may be configured for performing mapping/alignment of sequencing data, sorting/deduplicating of sequencing data, variant calling, and/or other types of secondary analysis. Though the FPGA 127a may be configured with engines for performing different types of secondary analysis, the FPGA 127a may be reconfigured for performing different types of secondary analysis (e.g., mapping/aligning, sorting/deduplication, variant calling, etc.). Each bitstream may be preconfigured with one or more engines for performing the corresponding secondary analysis. As the FPGA 127a may be assigned to the individual client process 210a, the reconfiguration of the FPGA 127a may be performed without delay that may be caused by other client processes accessing the same FPGA 127a. After the client process 210a has completed the secondary analysis of the sequencing data, the FPGA 127a may be available for being assigned to another client process by the daemon process 160.

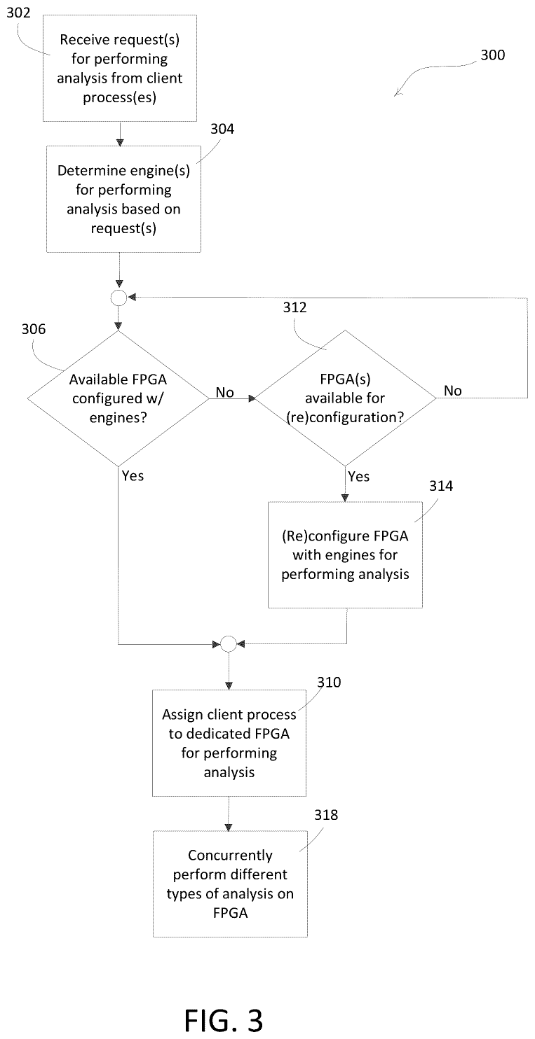

FIG. 3 is a flowchart depicting an example procedure 300 for performing analysis and processing requests from client processes. The one or more portions of the procedure 300 may be performed by one or more computing devices. For example, the one or more portions of the procedure 300 may be performed by one or more server devices, one or more sequencing devices, and/or one or more client devices. One or more portions of the procedure 300 may be stored in memory as computer-readable or machine-readable instructions that may be executed by a processor of the one or more computing devices. One or more portions of the procedure 300 may be performed by one or more subsystems operating on a client device, sequencing device, and/or a server device. For example, one or more portions of the procedure may be performed by a client process, a bioinformatics subsystem, and/or a scheduler subsystem, as described herein.

The procedure 300 may begin at 302. As shown in FIG. 3, at 302 the scheduler subsystem may receive one or more requests from one or more client processes. The requests may be received individually or concurrently for being processed by the bioinformatics subsystem. The requests may include a request for a dedicated FPGA for performing secondary analysis or tertiary analysis of sequencing data. There may be multiple FPGAs or FPGA boards on the computing system for being assigned to a corresponding client process for performance of the requested analysis. The request may include a type of secondary analysis or tertiary analysis to be performed. The request may include an identifier of an engine, or set of engines, to perform a type of secondary analysis or tertiary analysis. The request may include a version identifier of a version of software and/or engine to perform the secondary analysis or tertiary analysis. The request may include an identifier of the client process requesting the analysis.

At 304, the scheduler subsystem may determine an engine, or set of engines, for performing the requested secondary analysis or tertiary analysis. The scheduler subsystem may determine, at 306, whether there is an FPGA that is available that is configured with the engine, or set of engines, being requested. If an FPGA is available with the engines, or set of engines, for performing the secondary analysis or tertiary analysis for the client process, the scheduler subsystem may assign the client process to the FPGA for performing secondary analysis or tertiary analysis at 310. The assignment may be performed by instructing the daemon subsystem to perform the assignment and/or establish a connection between the client process and the FPGA (or one or more engines thereon) for servicing the requests. The FPGA may be assigned as a dedicated FPGA for performing different types of secondary analysis or tertiary analysis in response to requests from the assigned client process.

If, at 306, the scheduler subsystem determines that an FPGA with the proper configuration is unavailable, the scheduler subsystem may determine at 312 whether there are available FPGAs for being configured/reconfigured for servicing the requests of the client processor. If the FPGAs are determined to be unavailable for configuration/reconfiguration, the scheduler subsystem may cause the client process to continue to wait for an FPGA with the proper configuration for being assigned. If an FPGA is available for configuration/reconfiguration at 312, the scheduler subsystem may instruct the daemon process operating at the bioinformatics subsystem to configure/reconfigure the FPGA. The configuration/reconfiguration may be performed at 314 by loading a bitstream image to the FPGA for configuring one or more engines on the FPGA. The bitstream image may include a single instance comprising the configuration for multiple engines. For example, the multiple engines may comprise different engines (e.g., unzip engine, mapping engine, read alignment engine, sorting engine, dedup engine, zipping engine, haplotype assembly engine, haplotype alignment engine, read probability engine, etc.) for performing different types of secondary analysis (e.g., mapping/alignment, sorting, variant calling, etc.) or tertiary analysis. Each engine may occupy a different logical portion of the FPGA. Each engine may operate in a cluster of engines at the FPGA. After the FPGA has been configured/reconfigured at 314, the scheduler subsystem may assign the client process to the FPGA for performing secondary analysis or tertiary analysis at 310. The assignment may be performed by instructing the daemon subsystem to perform the assignment and/or establish a connection between the client process and the FPGA (or one or more engines thereon) for servicing the requests. The FPGA may be assigned as a dedicated FPGA for performing different types of secondary analysis in response to requests from the assigned client process. The client process may be assigned to one or more dedicated engines in the FPGA and a connection may be established for performing one or more types of secondary analysis.

At 318, the FPGA may be implemented to concurrently perform different types of secondary analysis or tertiary analysis. The FPGA may include a single instance comprising multiple engines configured for performing different types of secondary analysis or tertiary analysis. For example, the dedicated FPGA may be implemented to perform mapping/aligning and/or sorting/deduplication of portions of the sequencing data for the assigned client process, and concurrently perform variant calling on other portions of the sequencing data that has been previously processed via other types of secondary analysis. The FPGA may also, or alternatively, be implemented to concurrently perform secondary analysis and tertiary analysis. As secondary analysis or tertiary analysis is completed for each client process, the FPGAs may be reconfigured/reassigned to subsequent client processes for performing secondary analysis or tertiary analysis, as described herein.

Each client process having a dedicated FPGA may allow for the FPGA resources to be given priority to the requests for hardware acceleration of secondary and/or tertiary analysis from a particular client process. This level of priority may allow for a relatively faster completion of the secondary analysis for a given client process than when common resources on an FPGA may be shared across client processes. For example, a given client process may have requests for hardware acceleration of each type of secondary analysis serviced without delay that may be caused in common FPGA resources being leveraged by other client processes.

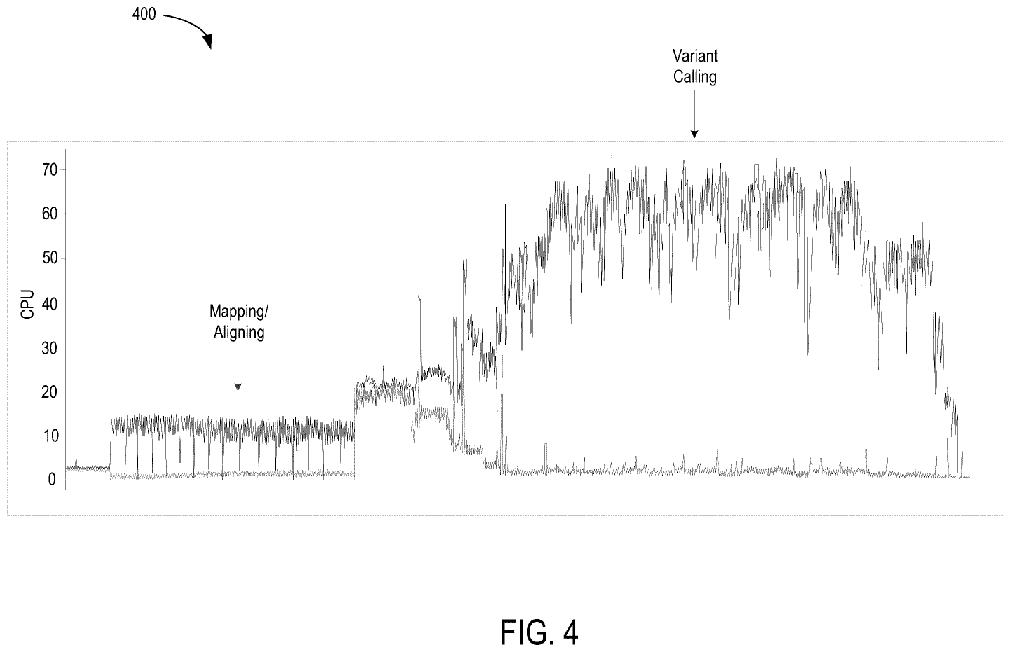

However, giving each client process access to a dedicated FPGA may be a less efficient use of total FPGA resources and/or CPU resources than when the resources on an FPGA are shared across multiple client processes. FIG. 4 is a graph 400 illustrating CPU usage as a percentage over time for performing different types of secondary analysis while each FPGA is assigned to a dedicated client process. As shown in FIG. 4, a relatively greater amount of CPU resources may be occupied when performing variant calling than when performing mapping/aligning. For example, the first portion of the graph 400 shows approximately ˜12% CPU consumption when performing mapping/aligning, while a second portion of the graph 400 shows approximately ˜65% CPU consumption when performing variant calling. Different engines, or sets of engines, for performing corresponding forms of secondary analysis may also occupy greater CPU resources than other engines, or sets of engines. For example, a mapping engine, read alignment engine, and/or a haplotype assembly engine that implements a graphing algorithm may occupy more resources than other engine types. As each of the FPGAs may be implementing the same engine at the same time, CPUs may be reserved for these peak periods, which may reduce the total CPU usage at other periods of time. Using the orthogonal mode in which each FPGA is assigned to a dedicated client process may create challenges in achieving a total CPU utilization over a threshold value (e.g., above 70-80%). This may be because each client process may be reserved nearly equal portions of CPU resources. Additionally, the FPGA usage for each FPGA may be inconsistent causing periods of time at which a given FPGA's utilization is relatively low.

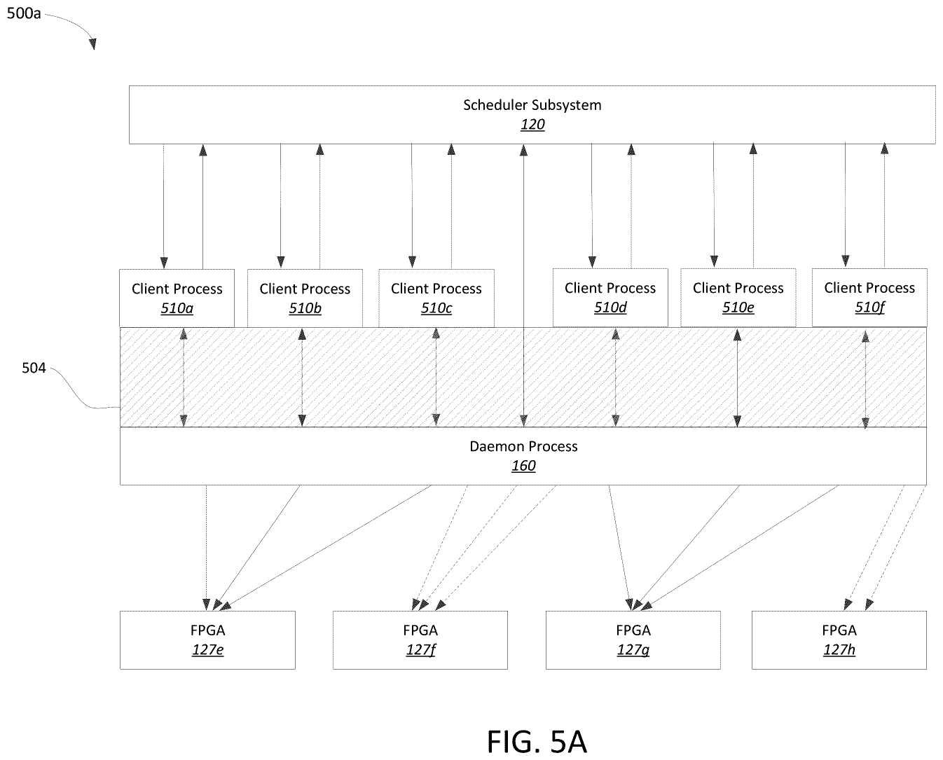

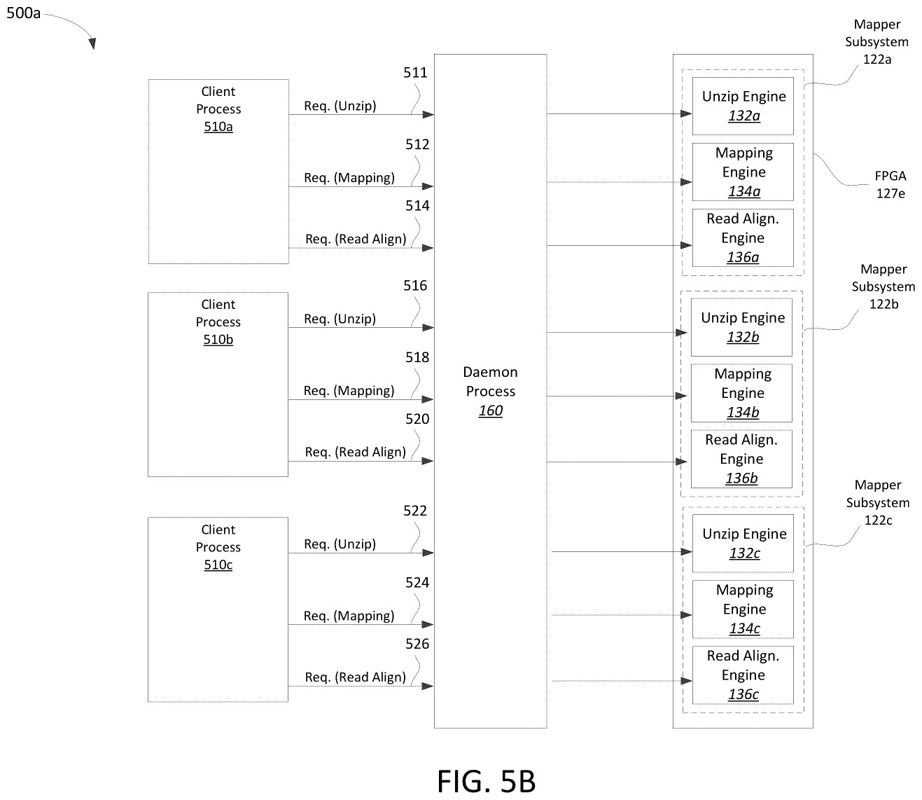

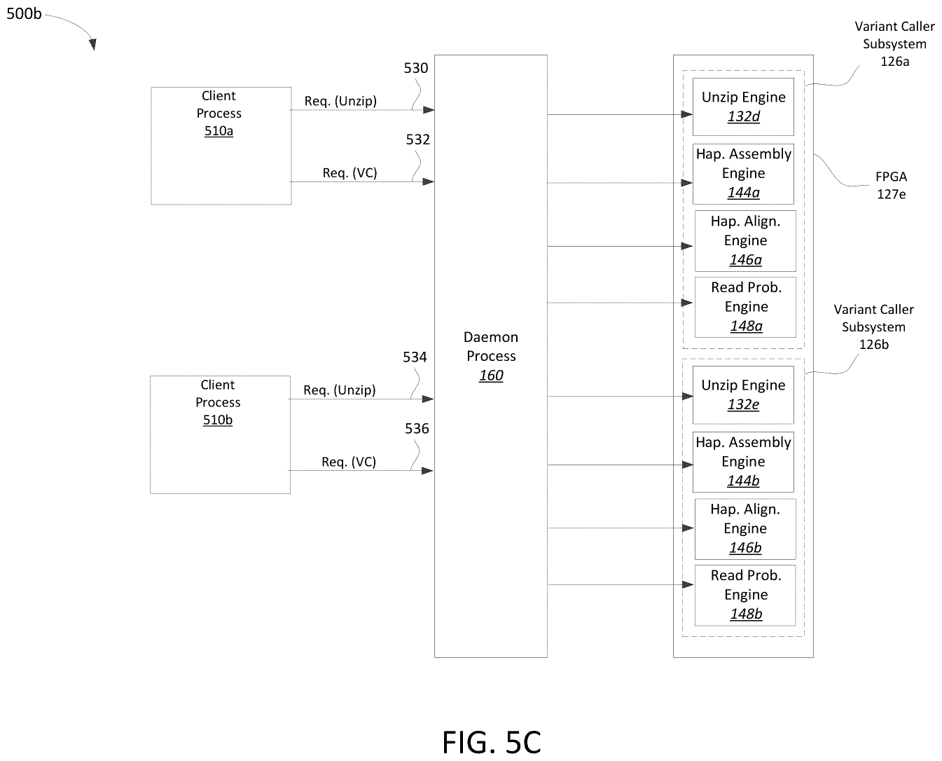

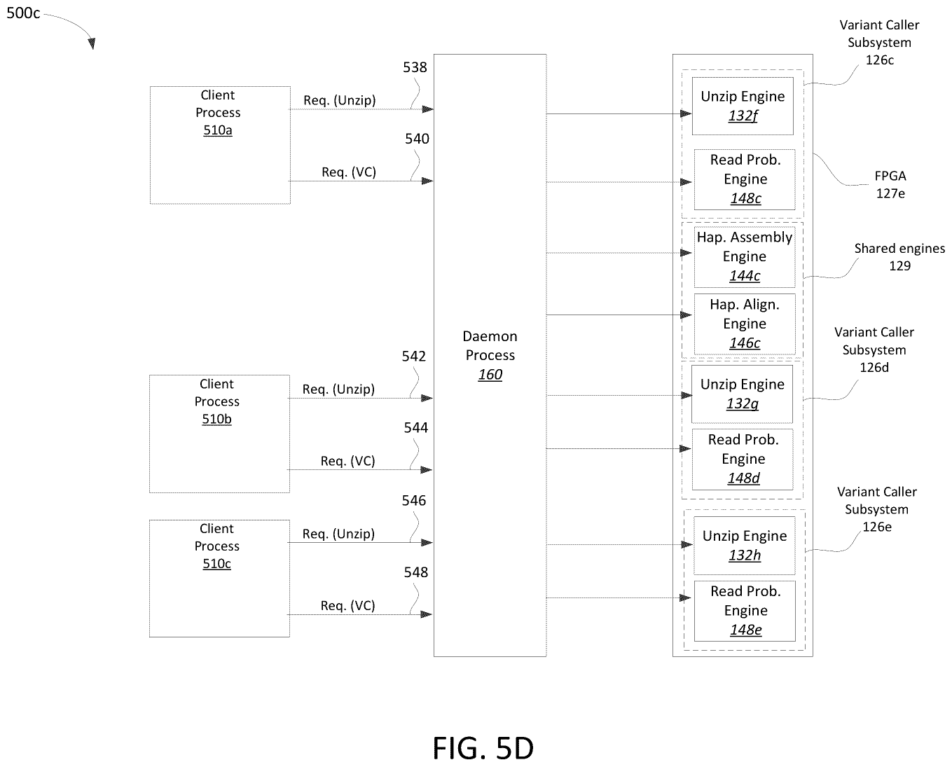

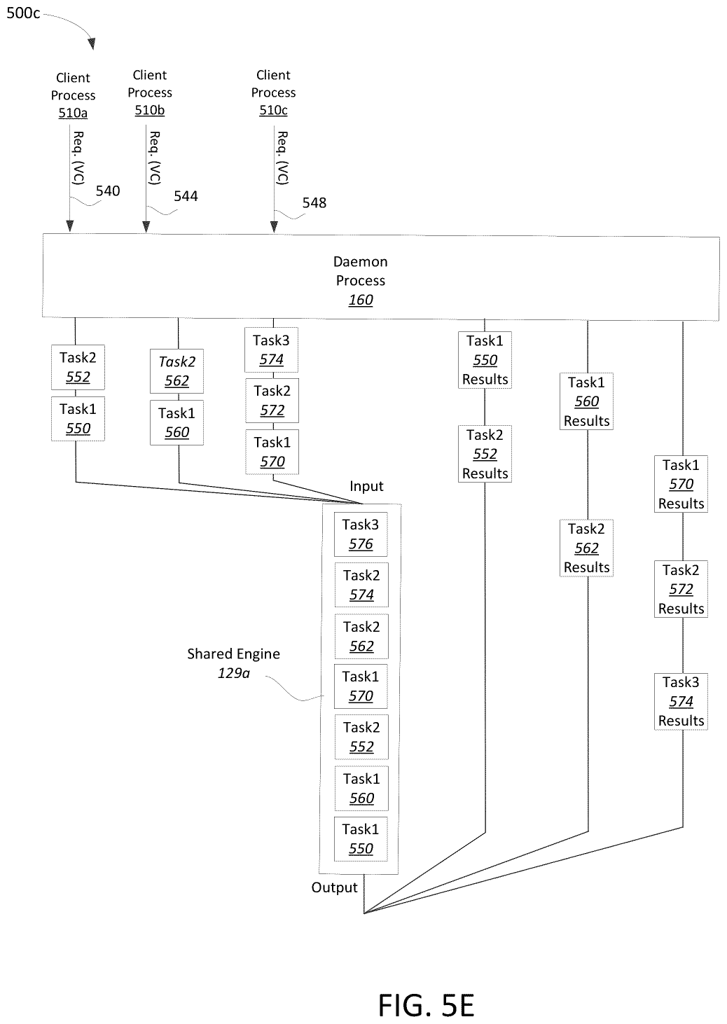

To better balance CPU resources and/or FPGA resources on average or at given periods of time, each FPGA may be shared across multiple client processes. FIG. 5A illustrates an example system 500a in which one or more FPGAs 127 may be shared by client processes at a given time. For example, as shown in FIG. 5A, each client process 510a, 510b, 510c, 510d, 510d, 510e, 510f (collectively referred to as client processes 510) may transmit a request to the scheduler subsystem 120 for accessing one or more engines on one of FPGAs 127e, 127f, 127g, 127h for performing secondary analysis of sequencing data. As each of the client processes 510 may share access to the FPGAs 127e, 127f, 127g, 127h for performing secondary analysis, the secondary analysis may be performed for the client processes 510 in a coordinated mode for coordinated the shared resources. In the coordinated mode, the same type of secondary analysis may be performed for multiple client processes 510 in parallel on a single FPGA.

The scheduler subsystem 120 may be in communication with the daemon process 160. When each of the FPGAs 127e, 127f, 127g, 127h are available, the daemon process 160 may communicate the availability of one or more FPGAs 127e, 127f, 127g, 127h to service the requests of the client process 510a to the scheduler subsystem 120. The scheduler subsystem 120 may cause the daemon process 160 to configure one or more of the FPGAs 127e, 127f, 127g, 127h to perform secondary analysis in response to the requests from the client processes 510. Each of the client processes 510 may be authorized by the scheduler subsystem 120 to communicate requests for performing hardware acceleration of secondary analysis to the daemon process 160 after an FPGA is properly configured. In the coordinated mode, the scheduler subsystem 120 may schedule each of the client processes 510 for which a given FPGA 127e, 127f, 127g, 127h has an engine, or set of engines, configured for performing the requested hardware acceleration of secondary analysis (e.g., mapping/aligning, sorting, deduplicating, variant calling, and/or another type of secondary analysis).

The scheduler subsystem 120 and identify the engines that are being requested by the client processes 510 for configuring each of the FPGAs 127e, 127f, 127g, 127h. The scheduler subsystem 120 may instruct the daemon process 160 to configure one or more FPGAs in response to the requests. In an example, the configuration of the FPGA 127e may be based on the requests from the client processes 510a, 510b, 510c and the configuration of FPGA 127g may be based on the requests from client processes 510d, 510e, 510f. The client processes 510 may each submit a request to the scheduler subsystem 120 for performing secondary analysis and include an identification of an engine, or set of engines, for performing the requested hardware acceleration of secondary analysis. The request may also include a version of the client process and/or the requested engine to be leveraged for performing the secondary analysis. The scheduler subsystem 120 may identify the engines and/or types of secondary analysis that are being requested by the client processes 510 and instruct the daemon process 160 to configure the FPGAs 127e, 127f, 127g, 127h for performing the requested hardware acceleration of secondary analysis. For example, each client process 510a, 510b, and 510c may request one or more engines for performing mapping/alignment of sequencing data. Each client process 510d, 510e, and 510f may request one or more engines for performing variant calling of sequencing data.

In response to the requests from client processes 510a, 510b, 510c, the daemon process 160 may identify or be notified (e.g., by the scheduler subsystem 120) of the configuration for the FPGA 127e with the engine, or set of engines, for performing the requested hardware acceleration of secondary analysis. The FPGA 127e may be configured with multiple instances of each engine, or set of engines, for processing the requests from client processes 510a, 510b, 510c. For example, the FPGA 127e may be configured with a separate instance of an engine, or set of engines, for performing the requested hardware acceleration of secondary analysis (e.g., mapping/alignment) of the sequencing data for each client process 510a, 510b, 510c. After the FPGA 127e is configured with the engine, or set of engines, for performing the secondary analysis requested by the client processes 510a, 510b, 510c, the client processes 510a, 510b, 510c may be given access by the scheduler subsystem 120 to establish a connection with the daemon process at 504. The daemon process 160 may establish the connection for each of the client processes 510a, 510b, 510c to an engine, or set of engines, of the FPGA 127e for servicing the requested hardware acceleration of secondary analysis. The FPGA 127e may be utilized to perform the requested mapping/alignment for each of the client processes 510a, 510b, 510c concurrently on the shared FPGA 127e.

In response to each of the client processes 510a, 510b, 510c transmitting a request to the daemon process 160, the daemon process 160 may establish a separate connection with each of the client processes 510a, 510b, 510c at 504. The connection may be established at 504 to allow each of the client processes 510a, 510b, 510c to read and/or write data to a respective connection. Each connection for a process may be assigned an amount and/or location of memory for processing sequencing data in response to the requests. As a part of the connections that are established at 504, the daemon process 160 may establish an individual stream for sending data to and/or receiving data from assigned engines on one or more FPGAs.

In response to the requests from client processes 510d, 510e, 510f, the daemon process 160 may identify or be notified (e.g., by the scheduler subsystem 120) that client processes are requesting the same engine, or set of engines, and configure the FPGA 127g with the engine, or set of engines, for performing the requested hardware acceleration of secondary analysis. For example, the FPGA 127g may be configured with a an engine, or set of engines, for performing the requested hardware acceleration of secondary analysis (e.g., variant calling) of the sequencing data for each client process 510d, 510e, 510f. After the FPGA 127g is configured with the engine, or set of engines, for performing the hardware acceleration of secondary analysis requested by the client processes 510e, 510f, 510g, the client processes 510d, 510e, 510f may be given access by the scheduler subsystem 120 to establish a connection with the daemon process 160 at 504. The daemon process 160 may establish the connection for each of the client processes 510d, 510e, 510f to a separate engine, or set of engines, of the FPGA 127g for servicing the requested hardware acceleration of secondary analysis. Having separate instances of each engine, or set of engines, processing the requests of each of the client processes 510d, 510e, 510f may allow the requested hardware acceleration of secondary analysis (e.g., variant calling) to be performed for each of the client processes 510d, 510e, 510f concurrently on the FPGA 127g.

As illustrated in FIG. 5A, in the coordinated mode the same type of secondary analysis may be performed for multiple client processes 510 concurrently on a single FPGA, while the same or different types of secondary analysis may be performed for other client processes 510 in parallel on one or more other FPGAs. The use of multiple FPGAs, each processing the requests of multiple client processes, may allow for secondary analysis to be performed concurrently on the FPGAs to balance the FPGA and CPU resources and to optimize processing for servicing the client processes. Though FPGA 127e and FPGA 127g are described as being configured for performing secondary analysis requested by client processes 510, FPGA 127f and FPGA 127h may be further configured with engines for similarly performing secondary analysis of additional client processes. For example, assuming a configuration of four FPGAs 127e, 127f, 127g, 127h, and each FPGA being configured with three to four separate instances of one or more engines, secondary analysis may be performed concurrently for twelve to sixteen client processes. However, it will be understood that the number of FPGAs and/or the number of instances of engines configured on each FPGA may vary.

Each of the FPGAs 127e, 127f, 127g, 127h may initially be configured for a first type of secondary analysis (e.g., decompressing and/or mapping/aligning sequencing data), as each of the client processes 510 may be requesting hardware acceleration for the same type of secondary analysis (e.g., using the same or different versions of software) upon initial startup. As FPGA resources become available, an available FPGA may be reconfigured for performing another type of secondary analysis (e.g., variant calling) for servicing additional requests of client processes, or the same type of secondary analysis in another version of software. For example, the daemon process 160 may assign the client process 510a to an engine, or set of engines, configured on FPGA 127e for performing mapping/alignment and, after the mapping/alignment is completed for the client process 510a and the client process 510a requests one or more engines for performing variant calling, the daemon process may assign the client process 510a to an engine, or set of engines, configured on the FPGA 127g for performing variant calling of the mapped/aligned sequencing data.

The daemon process 160 may configure each of the FPGAs 127e, 127f, 127g, 127h for servicing the requests from the assigned client process. For example, the daemon process 160 may cause the loadable kernel driver 162 to load one or more engines, via a bitstream image, into each FPGA for processing the requests from a dedicated client process. Each FPGA 127e, 127f, 127g, 127h may comprise a partial reconfiguration bitstream that is capable of enabling the FPGA to service requests for hardware acceleration of secondary analysis from the client processes. Each of the FPGAs 127e, 127f, 127g, 127h may be loaded with a distinct set of engines required to perform secondary analysis, or they may be the same. At different times, as different portions of the secondary analysis progress, each of the FPGAs 127e, 127f, 127g, 127h may be configured and/or reconfigured with different engines to service the current requirements of the clients.

FIG. 5B illustrates a portion of the system 500a and shows an example configuration of an FPGA 127e (e.g., FPGA 127e shown in FIG. 5A) for servicing requests for hardware acceleration of secondary analysis from assigned client processes 510a, 510b, 510c. Other FPGAs may be similarly configured or differently configured for servicing requests for hardware acceleration of secondary analysis from other client processes. As shown in FIG. 5B, the client process 510a may be assigned to an engine, or set of engines, configured on the FPGA 127e by the daemon process 160. For example, the client process 510a may be assigned to an engine, or set of engines, of a mapper subsystem 122a for performing mapping/alignment of sequencing data. The client process 510b may be assigned to an engine, or set of engines, configured on the FPGA 127e by the daemon process 160. For example, the client process 510b may be assigned to an engine, or set of engines, of a mapper subsystem 122b for performing mapping/alignment of sequencing data. The client process 510c may be assigned to an engine, or set of engines, configured on the FPGA 127e by the daemon process 160. For example, the client process 510c may be assigned to an engine, or set of engines, of a mapper subsystem 122c for performing mapping/alignment of sequencing data.

The client processes 510a, 510b, 510c may each send one or more requests to the daemon process 160 for requesting hardware acceleration of one or more types of secondary analysis to be performed on sequencing data. For example, the client process 510a may send a request 511, 512, 514 for accessing each engine or set of engines 132a, 134a, 136a of the mapper subsystem 122a. Each request 511, 512, 514 may identify a corresponding engine 132a, 134a, 136a for performing the requested task. The client processes 510b may send a request 516, 518, 520 for accessing each engine or set of engines 132b, 134b, 136b of the mapper subsystem 122b. Each request 516, 518, 520 may identify a corresponding engine 132b, 134b, 136b for performing the requested task. The client processes 510c may send a request 522, 524, 526 for accessing each engine or set of engines 132c, 134c, 136c of the mapper subsystem 122c. Each request 522, 524, 526 may identify a corresponding engine 132c, 134c, 136c for performing the requested task. Though individual requests are illustrated as being transmitted for a respective engine, a single request may be sent to identify a set of engines for performing a corresponding type of secondary analysis and the daemon process 160 may assign a given client process to a set of engines in response to the request.

The FPGA 127e may be configured with engines for performing multiple instances of the same types of secondary analysis. For example, the FPGA 127e may include multiple instances of mapper subsystem 122a, 122b, 122c that are each configured to map/align the reads in the sequencing data from an assigned client process. As shown in FIG. 5B, the FPGA 127e may be configured with a first instance of a mapper subsystem 122a that includes one or more of an unzip engine 132a, a mapping engine 134a, and/or a read alignment engine 136a. The FPGA 127e may be configured with a second instance of a mapper subsystem 122b that includes one or more of an unzip engine 132b, a mapping engine 134b, and/or a read alignment engine 136b. The FPGA 127e may be configured with a third instance of a mapper subsystem 122c that includes one or more of an unzip engine 132c, a mapping engine 134c, and/or a read alignment engine 136c. It will be understood that more or fewer engines may be configured to perform the mapping and/or alignment of the sequencing data, as described herein. Similarly, it will be understood that an FPGA may be configured with more or fewer engines for being assigned to a greater or lesser number of client processes.

The engines that are configured on the FPGA 127e may be configured via a bitstream image stored on disk 123 and loaded onto the FPGA 127e via RAM 125. The bitstream image may be preconfigured with a predefined number of engines and/or engine types for performing secondary analysis to support up to a predefined number of concurrent client processes. For example, the bitstream may be preconfigured with multiple instances of the mapper subsystems 122a, 122b, 122c that are each configured to map/align the reads in the sequencing data of an assigned client process. The bitstream being preconfigured with multiple instances of an engine, or set of engines, (e.g., engines of the mapper subsystem 122a, the mapper subsystem 122b, and/or the mapper subsystem 122c) may allow the FPGA 127e to perform the same requested hardware acceleration of secondary analysis for multiple client processes concurrently.

As shown in FIG. 5B, the FPGA 127e may be configured with different sets or collections of engines in multiple instances operating the same secondary analysis on different subsets of hardware resources on the same FPGA 127e. Each engine, or set of engines, may be configured using different logical portions of the FPGA 127e to perform the same type of secondary analysis. The FPGA 127e may include an array of programmable logic blocks and a hierarchy of reconfigurable interconnects that may be programmed by the bitstream images. Each engine, or set of engines, may be configured in a different cluster on the FPGA 127e to support the same types of secondary analysis. In the example shown in FIG. 5B, the FPGA 127e may be configured with different logical portions for the engines, or a set of the engines, of the mapper subsystem 122a, 122b, and 122c. Each engine, or set of engines, may be operating using the same or different version of software and/or support client processes operating on the same or different versions of software.

Each mapper subsystem 122a, 122b, 122c may receive and process a separate file that includes sequencing data as input and/or generate a separate file as output. For example, each mapper subsystem 122a, 122b, 122c may receive sequencing data in a separate file (e.g., a FASTQ file) that corresponds to the assigned client process 510a, 510b, 510c and perform secondary analysis (e.g., mapping/aligning) on the sequencing data in the file. The sequencing data may be loaded from disk 123. Each mapper subsystem 122a, 122b, 122c may generate a separate output file that includes mapped and/or aligned reads of the sequencing data received as input. For example, each mapper subsystem 122a, 122b, 122c may generate a separate file (e.g., a BAM file or CRAM file) that corresponds to the assigned client process 510a, 510b, 510c for being stored in another location (e.g., on disk 123) for being reloaded for performing subsequent secondary analysis.

After one of the mapper subsystems 122a, 122b, 122c has finished performing secondary analysis for an assigned client process 510a, 510b, 510c, the mapper subsystem 122a, 122b, 122c that has completed the requested analysis may be available for reassignment to another client process. Each of the engines, or set of engines, in the mapper subsystem 122a, 122b, 122c that has completed may be reassigned to another client process requesting the hardware acceleration of secondary analysis for which the mapper subsystem is configured.

The scheduler subsystem 120 and/or the daemon process 160 may continue to assign the mapper subsystems 122a, 122b, 122c to client processes until a triggering event is met for reconfiguration of the FPGA 127e. For example, the triggering event may include an indication that each of the client processes has completed the secondary analysis for which the FPGA 127e is configured and/or a predefined period of time has elapsed since the completion. The triggering event may include an indication that each of the client processes has been assigned for performing the secondary analysis for which the FPGA 127e is configured and/or a predefined period of time has elapsed since the assignments. The triggering event may include an indication that less than a threshold number of client processes have requested the hardware acceleration of secondary analysis for which the FPGA 127e is configured (e.g., when one or more other FPGAs are configured for performing the same type of secondary analysis). The triggering event may be identified when a new client process has requested support for a type of secondary/tertiary analysis acceleration unsupported by the currently loaded bitstream. The triggering event may be identified when the scheduler subsystem 120 or a client has explicitly requested the loading of a different configuration. The triggering events may be determined at the scheduler subsystem 120 based on the information in the requests received by the scheduler subsystem 120.

After the triggering event is identified for reconfiguring an available FPGA, the daemon process 160 may reconfigure (e.g., at the request of the scheduler subsystem 120) the available FPGA with another bitstream for performing another type of secondary analysis (e.g., variant calling), or for performing the same type of secondary analysis utilizing another version of software. For example, the FPGA 127e may be configured (e.g., initially or reconfigured) for performing variant calling for multiple client processes.

FIG. 5C illustrates a portion of the system 500b and shows another example configuration of an FPGA 127e (e.g., FPGA 127e shown in FIG. 5A) for servicing requests for secondary analysis from assigned client processes 510a, 510b. Other FPGAs may be similarly configured or differently configured for servicing requests for hardware acceleration of secondary analysis from other client processes. As shown in FIG. 5C, the client process 510a may be assigned to an engine, or set of engines, configured on the FPGA 127e by the daemon process 160. For example, the client process 510a may be assigned to an engine, or set of engines, of a variant caller subsystem 126a for performing variant calling of mapped/aligned sequencing data. The client process 510b may be assigned to an engine, or set of engines, configured on the FPGA 127e by the daemon process 160. For example, the client process 510b may be assigned to an engine, or set of engines, of a variant caller subsystem 126b for performing variant calling of mapped/aligned sequencing data.

The client processes 510a, 510b may each send one or more requests to the daemon process 160 for requesting hardware acceleration of one or more types of secondary analysis to be performed on sequencing data. For example, the client process 510a may send a request 530, 532 for accessing each engine or set of engines 132d, 144a, 146a, 148a of the variant caller subsystem 126a for performing variant calling of mapped/aligned sequencing data. Each request 530, 532, may identify an engine, or set of engines, in the variant caller subsystem 126a for performing the requested task. The client processes 510b may send a request 534, 536 for accessing each engine or set of engines 132e, 144b, 146b, 148b of the variant caller subsystem 126b for performing variant calling of mapped/aligned sequencing data. Each request 534, 536 may identify an engine, or set of engines, for performing the requested function. Though individual requests are illustrated as being transmitted for a respective engine or for requesting hardware acceleration of a type of secondary analysis that corresponds to an engine or set of engines, a single request may be sent to identify one or more engines for performing a corresponding type of secondary analysis and the daemon process 160 may assign a given client process to one or more engines in response to the request.

The FPGA 127e may be configured/reconfigured with engines for performing multiple instances of the same types of secondary analysis. For example, the FPGA 127e may include multiple instances of variant caller subsystem 126a, 126b that are each configured to perform variant calling on the sequencing data from an assigned client process. As shown in FIG. 5C, the FPGA 127e may be configured with a first instance of a variant caller subsystem 126a that includes one or more of an unzip engine 132d, a haplotype assembly engine 144a, a haplotype alignment engine 146a, and/or a read probability engine 148a. The FPGA 127e may be configured with a second instance of variant caller subsystem 126b that includes one or more of an unzip engine 132e, a haplotype assembly engine 144b, a haplotype alignment engine 146b, and/or a read probability engine 148b. It will be understood that more or fewer engines may be configured to perform the variant calling of the sequencing data, as described herein. Similarly, it will be understood that an FPGA may be configured with more or fewer engines for being assigned to a greater or lesser number of client processes.

The engines that are configured on the FPGA 127e may be configured via a bitstream image stored on disk 123 and loaded onto the FPGA 127e via RAM 125. The bitstream image may be preconfigured with a predefined number of engines and/or engine types for performing secondary analysis to support up to a predefined number of concurrent client processes. For example, the bitstream may be preconfigured with one or more engines of the variant caller subsystems 126a, 126b that are each configured perform variant calling for the sequencing data of an assigned client process. The bitstream being preconfigured with multiple instances of engines (e.g., engines of the variant caller subsystem 126a and the variant caller subsystem 126b) on the FPGA 127e in order to accelerate multiple concurrent clients each of which are performing the same type of secondary analysis.

Each instance of the variant caller subsystems 126a, 126b and/or the engines therein may be configured to accelerate the same type of secondary analysis (e.g., variant calling) on different sequencing data for different client processes. In the example shown in FIG. 5C, the FPGA 127e may be configured with different logical portions of clusters for the engines, or a set of the engines, of the variant caller subsystem 126a, 126b. Each engine, or set of engines, may be operating using the same or different version of software and/or support client processes operating on the same or different versions of software.

Each variant caller subsystem 126a, 126b may receive and process separate input data (e.g., a separate input file) that includes mapped/aligned sequencing data as input and/or generate separate output data (e.g., a separate output file) as output. For example, each variant caller subsystem 126a, 126b may receive sequencing data in a separate file (e.g., a BAM or CRAM file) that corresponds to the assigned client process 510a, 510b and perform secondary analysis (e.g., variant calling) on the sequencing data in the file in response to one or more requests. The sequencing data may be loaded from disk 123. Each variant caller subsystem 126a, 126b may generate a separate output file that includes mapped and/or aligned reads of the sequencing data received as input. For example, each variant caller subsystem 126a, 126b may generate a separate file (e.g., a VCF or gVCF file) that corresponds to the assigned client process 510a, 510b for being stored in another location (e.g., on disk 123) for being used in analyzing the variant calls and/or sequencing data.

After client process 510a, 510b is through using the hardware acceleration of one of the variant caller subsystems 126a, 126b, the variant caller subsystem 126a, 126b that has completed the requested analysis may be available for reassignment to another client process. Each of the engines, or set of engines, in the variant caller subsystems 126a, 126b that has completed may be reassigned to another client process requesting the secondary analysis for which the variant caller subsystem is configured.

The scheduler subsystem 120 and/or the daemon process 160 may continue to assign the variant caller subsystems 126a, 126b to client processes until a triggering event is met for reconfiguration of the FPGA 127e. For example, the triggering events may be similar to those described elsewhere herein (e.g., an indication that each of the client processes has completed the secondary analysis for which the FPGA 127e is configured; a predefined period of time; an indication that each of the client processes has been assigned for performing the secondary analysis for which the FPGA 127e is configured; an indication that less than a threshold number of client processes have requested the hardware acceleration of secondary analysis for which the FPGA 127e is configured; etc.).

After a triggering event has been identified for reconfiguring an available FPGA, the daemon process may be caused to reconfigure the available FPGA with another bitstream for performing another type of secondary analysis, or for performing the same type of secondary analysis utilizing another version of software.

One or more engines configured on the FPGA 127e for performing secondary analysis may be shared by multiple client processes. FIG. 5D illustrates a portion of the system 500c and shows another example configuration of an FPGA 127e (e.g., FPGA 127e shown in FIG. 5A) for servicing requests for hardware acceleration of secondary analysis from assigned client processes 510a, 510b, 510c using one or more shared engines 129. Other FPGAs may be similarly configured or differently configured for servicing requests for hardware acceleration of secondary analysis from other client processes using shared engines. As shown in FIG. 5D, the client process 510a may be assigned to an engine, or set of engines, configured on the FPGA 127e by the daemon process 160. For example, the client process 510a may be assigned to an engine, or set of engines, of a variant caller subsystem 126c for performing variant calling of mapped/aligned sequencing data. The client process 510b may be assigned to an engine, or set of engines, of a variant caller subsystem 126d for performing variant calling of mapped/aligned sequencing data. The client process 510c may be assigned to an engine, or set of engines, of a variant caller subsystem 126e for performing variant calling of mapped/aligned sequencing data.

Each of the client processes 510a, 510b, 510c may also be assigned to one or more shared engines 129. The one or more shared engines 129 may be independent of or a part of the variant caller subsystems 126c, 126d, 126e. For example, the shared engines 126c may occupy independent and/or overlapping resources on the FPGA 127e.

The client processes 510a, 510b, 510c may each send one or more requests to the daemon process 160 for requesting hardware acceleration of one or more types of secondary analysis to be performed on sequencing data. For example, the client process 510a may send a request 538, 540 for accessing each engine or set of engines 132f, 148c of the variant caller subsystem 126c for performing variant calling of mapped/aligned sequencing data. Each request 538, 540, may identify an engine, or set of engines, in the variant caller subsystem 126c for performing the requested function. The client process 510b may send a request 542, 544 for accessing each engine or set of engines 132g, 148d of the variant caller subsystem 126d for performing variant calling of mapped/aligned sequencing data. Each request 542, 544 may identify an engine, or set of engines, for performing the requested function. The client process 510c may send a request 546, 548 for accessing each engine or set of engines 132h, 148e of the variant caller subsystem 126e for performing variant calling of mapped/aligned sequencing data. Each request 546, 548 may identify an engine, or set of engines, for performing the requested function. Though individual requests are illustrated as being transmitted for a respective engine or for requesting hardware acceleration of a type of secondary analysis that corresponds to an engine or set of engines, a single request may be sent to identify one or more engines for performing a corresponding type of secondary analysis and the daemon process 160 may assign a given client process to one or more engines in response to the request.

Different types of tasks may be performed on the FPGA 127e in response to the requests from the client processes. For example, certain tasks may be capable of being performed on one or more shared engines 129 on the FPGA 127e that are capable of sharing resources among tasks for completing the requested hardware acceleration of secondary analysis. Tasks may also, or alternatively, be performed on one or more dedicated engines (e.g., unzip engine 132f, 132g, 132h and/or read probability engine 148c, 148d, 148e) on the FPGA 127e. Dedicated engines (e.g., unzip engine 132f, 132g, 132h and/or read probability engine 148c, 148d, 148e) may have data associated with a particular client inside the engine.

In response to a request, the daemon process 160 may assign one or more of the client processes 510a, 510b, 510c to one or more of the dedicated engines. Each dedicated engine may enter a state that is associated with the assigned client process 510a, 510b, 510c. For example, the unzip engine 132f and/or the read probability engine 148c may be dedicated engines that may be stateful for being exclusively assigned to the client process 510a. The unzip engine 132f and/or the read probability engine 148c may each occupy an area of the FPGA 127e that is reserved exclusively for the client process 510a until completion of tasks for the client process 510a while the client process 510a is assigned to the respective engine. The unzip engine 132f and/or the read probability engine 148c may each have data stored in portions of the FPGA 127e that are associated with the client process 510a for performing the respective tasks of each engine.

The unzip engine 132g and/or the read probability engine 148c may be dedicated engines that may be stateful for being exclusively assigned to the client process 510b. The unzip engine 132g and/or the read probability engine 148d may each occupy an area of the FPGA 127e that is reserved exclusively for the client process 510b until completion of tasks for the client process 510b while the client process 510b is assigned to the respective engine. The unzip engine 132g and/or the read probability engine 148d may each have data stored in portions of the FPGA 127e that are associated with the client process 510b for performing the respective tasks of each engine.

The unzip engine 132h and/or the read probability engine 148e may be dedicated engines that may be stateful for being exclusively assigned to the client process 510c. The unzip engine 132h and/or the read probability engine 148e may each occupy an area of the FPGA 127e that is reserved exclusively for the client process 510c until completion of tasks for the client process 510c while the client process 510c is assigned to the respective engine. The unzip engine 132h and/or the read probability engine 148e may each have data stored in portions of the FPGA 127e that are associated with the client process 510c for performing the respective tasks of each engine. Though the unzip engine and the read probability engines are provided as examples of dedicated engines that may occupy a state that is associated with a given client process, it will be understood that other types of client engines configured for performing other tasks or types of secondary analysis may be similarly configured on an FPGA.

As described herein, each of the client processes 510a, 510b, 510c may be assigned to one or more shared engines 129 for performing different types of secondary analysis. In response to a request, the daemon process 160 may assign one or more of the client processes 510a, 510b, 510c to one or more of the shared engines 129. For example, in response to each of the requests 540, 544, 548 received from the respective client processes 510a, 510b, 510c, the daemon process 160 may determine that each of the client processes are to be assigned to the shared engines 129. The shared engines 129 may be engines that may be implemented for performing variant calling. For example, the shared engines 129 may include haplotype assembly engine 144c and/or haplotype alignment engine 146c. Though, other engines configured for performing secondary analysis (e.g., mapping/aligning, sorting, and/or variant calling) may similarly be configured as shared engines.

The daemon process 160 may generate a set of tasks that may be sent to the shared engines 129 for being processed at the shared engines 129 for performing secondary analysis. FIG. 5E illustrates a portion of the system 500d and shows an example configuration of the daemon process 160 and tasks that may be processed by a shared engine 129a configured on the FPGA 127e. As shown in FIG. 5E, the requests 540, 544, 548 may be received by the daemon process 160. The requests 540, 544, 548 may include a request for hardware acceleration of variant calling to be performed on mapped/aligned sequencing data. However, requests for hardware acceleration of other types of secondary analysis (e.g., mapping/aligning, sorting, variant calling, and/or other types of secondary analysis) may be similarly processed on a shared engine.

The daemon process 160 may generate tasks for each request 540, 544, 548 received from a client process 510a, 510b, 510c. The tasks may be generated by time slicing the data retrieved for each request 540, 544, 548 from a client process 510a, 510b, 510c. The data retrieved from each request 540, 544, 548 may be time-sliced for a predefined period of time (e.g., 2 ms, 4 ms, etc.). The time slicing may result in each request being chunked into smaller tasks for being processed in smaller, more manageable, portions at the shared engine 129a. For example, tasks 550, 552 may be hardware tasks generated for performing secondary analysis at the FPGA 127e on sequencing data for client process 510a. Tasks 560, 562 may be hardware tasks generated for performing secondary analysis at the FPGA 127e on sequencing data for client process 510b. Tasks 570, 572, 574 may be hardware tasks generated for performing secondary analysis at the FPGA 127e on sequencing data for client process 510c. Each task may be tagged with an identifier of the client process from which the original request was received for performing hardware acceleration of the secondary analysis, such that the results of the secondary analysis may be returned to the proper client process or sent to downstream engines (e.g., shared engines or dedicated engines assigned to the client process identified by the tags).

The daemon process 160 may send the tasks on a stream for each connection to the shared engine 129a. The shared engine 129a may receive one or more tasks concurrently as input. For example, the shared engine 129a may receive the tasks on each of the streams in the established connections to the client processes 510a, 510b, 510c. The tasks may be processed serially or in parallel at the shared engine 129a. In one example, the shared engine 129a may process each task atomically, such that the processing of each task may be performed sequentially.

The shared engine 129a may output the results of each task and return the results to the daemon process 160 for coordinating the return to the appropriate client processes 510a, 510b, 510c. The results may be returned on the stream established for the connection to each client processes 510a, 510b, 510c. The results that are output for each task that has been completed may include the same tag as the task that was received as input at the shared engine 129a, such that the daemon process 160 may use tags to route the results to proper client process 510a, 510b, 510c or downstream engines (e.g., shared engines or dedicated engines assigned to the client process identified by the tags).

Shared engine 129a is provided as an example to illustrate the idea that any shared engine on an FPGA may be similarly configured and/or operate in a similar manner. Referring again to FIG. 5D, though individual engines (e.g., haplotype assembly engine 144c and haplotype alignment engine 146c) are provided as examples of shared engines 129, other engines may be configured and/or operate as described herein. Similarly, though individual engines (e.g., unzip engines and read probability engines) are provided as examples of dedicated engines, other engines may be configured and/or operate as described herein.

As shown in FIG. 5D, the FPGA 127e may be configured/reconfigured with shared engines 129 and/or dedicated engines for performing the same types of secondary analysis. For example, the FPGA 127e may include multiple instances of variant caller subsystem 126c, 126d, 126e that are each configured to perform variant calling on the sequencing data from an assigned client process. The FPGA 127e may be configured with a first instance of a variant caller subsystem 126c, a second instance of variant caller subsystem 126d, and a third instance of variant caller subsystem 126d. It will be understood that more or fewer instances may be configured on the FPGA 127e and/or each instance may include more or fewer engines. The number of engines and/or subsystems may depend on resources (e.g., CPU and/or FPGA resources) required to efficiently run each client's secondary analysis. The implementation of one or more shared engines 129 may allow for additional client processes (e.g., client process 510c) to be assigned for performance of concurrent secondary analysis at the FPGA 127e.

The engines that are configured on the FPGA 127e may be configured via a bitstream image stored on disk 123 and loaded onto the FPGA 127e via RAM 125. The bitstream image may be preconfigured with a predefined number of engines and/or engine types for performing secondary analysis to support up to a predefined number of concurrent client processes. For example, the bitstream may be preconfigured with one or more engines of the variant caller subsystems 126a, 126b that are each configured perform variant calling for the sequencing data of an assigned client process. The bitstream being preconfigured with multiple engines (e.g., dedicated engines and/or shared engines) may allow the FPGA 127e to perform the requested hardware acceleration of secondary analysis for multiple client processes concurrently.

The dedicated engines and/or subsystems that are configured on the FPGA 127e may be preconfigured for the same type of secondary analysis. For example, each instance of the variant caller subsystems 126c, 126d, 126e and/or the engines therein may be configured to perform the same type of secondary analysis (e.g., variant calling) on different sequencing data for different client processes. In the example shown in FIG. 5D, the FPGA 127e may be configured with different logical portions of clusters for the engines, or set of the engines, of the variant caller subsystems 126c, 126d, 126e. Each engine, or set of engines, may be operating using the same or different version of software and/or support client processes operating on the same or different versions of software. The FPGA 127e may also be configured with different logical portions of clusters for each of the shared engines 129. However, the shared engines 129 may each share the FPGA resources, as described herein.

Each variant caller subsystem 126c, 126d, 126e may receive and process separate input data (e.g., a separate input file) that includes mapped/aligned sequencing data as input. For example, each variant caller subsystem 126c, 126d, 126e may receive sequencing data in a separate file (e.g., a BAM or CRAM file) that corresponds to the assigned client process 510a, 510b, 510c and perform secondary analysis (e.g., variant calling) on the sequencing data in the file in response to one or more requests. The initially received files may be decompressed by each of the unzip engines 132f, 132g, 132h, respectively. The unzip engines 132f, 132g, 132h may be dedicated engines capable of exclusively processing the sequencing data from each of the respectively assigned client process 510a, 510b, 510c utilizing dedicated resources on the FPGA 127e. The daemon process 160 may coordinate the sending of the decompressed data for each of the client processes 510a, 510b, 510c to the shared engines 129 for performing haplotype assembly and/or haplotype alignment. The shared engines 129 may each utilize shared resources on the FPGA 127e. Each of the shared engines may receive sequencing data as input from a client process 510a, 510b, 510c or an upstream engine and generate an output for being returned to the client process or a downstream engine. For example, the results of the processing performed by the shared engines 129 may be provided to the daemon process 160 for being provided to a client process 510a, 510b, 510c or a downstream engine. The sequencing data for each client process 510a, 510b, 510c may be provided to a respectively assigned read probability engine 148c, 148d, 148e. The read probability engines 148c, 148d, 148e may be dedicated engines capable of exclusively processing the sequencing data from each of the respectively assigned client process 510a, 510b, 510c utilizing dedicated resources on the FPGA 127e. Each read probability engine 148c, 148d, 148e may generate a separate output data (e.g., a VCF or gVCF file) that corresponds to the assigned client process 510a, 510b, 510c for being stored in another location (e.g., on disk 123) for being used in analyzing the variant calls and/or sequencing data.

After one of the variant caller subsystems 126c, 126d, 126e has finished performing secondary analysis for an assigned client process 510a, 510b, 510c the variant caller subsystem 126c, 126d, 126e that has completed the requested analysis may be available for reassignment to another client process. Each of the engines, or set of engines, in the variant caller subsystems 126c, 126d, 126e that has completed may be reassigned to another client process requesting the hardware acceleration of secondary analysis for which the variant caller subsystem is configured. The variant caller subsystems 126c, 126d, 126e may utilize dedicated resources on the FPGA 127e that are reallocated. The shared engines 129 may utilize shared resources and be assigned by the daemon process 160 to concurrent client processes requesting the hardware acceleration of the type of secondary analysis for which the shared engines 129 are configured.

The scheduler subsystem 120 and/or the daemon process 160 may continue to assign the variant caller subsystems 126c, 126d, 126e and the shared engines 129 to client processes until a triggering event is met for reconfiguration of the FPGA 127e. For example, the triggering events may be similar to those described elsewhere herein (e.g., an indication that each of the client processes has completed the secondary analysis for which the FPGA 127e is configured; a predefined period of time; an indication that each of the client processes has been assigned for performing the secondary analysis for which the FPGA 127e is configured; an indication that less than a threshold number of client processes have requested the hardware acceleration of secondary analysis for which the FPGA 127e is configured; etc.).

Referring again to FIG. 5A, though examples are provided for the configuration and/or reconfiguration of the FPGA 127e, other FPGAs (e.g., FPGAs 127f-127h) may be similarly configured or reconfigured for processing requests from multiple client processes (e.g., corresponding assigned client processes 510b-510f). Additionally, it will be understood that other arrangements of engines may be configured on an FPGA for performing mapping/alignment of sequencing data, sorting/deduplicating of sequencing data, variant calling, and/or other types of secondary analysis in response to requests from multiple client processes. As the FPGAs 127e-127h may each be assigned to multiple client processes, and FPGAs may be configured for performing different types of secondary analysis (e.g., mapping/aligning, sorting, deduplicating, variant calling, etc.), the utilization of the FPGA resources and/or CPU resources may be increased or optimized. For example, a scheduling system could automatically monitor and adjust allocation of processes to CPUs and FPGAs in order to maximize throughput.

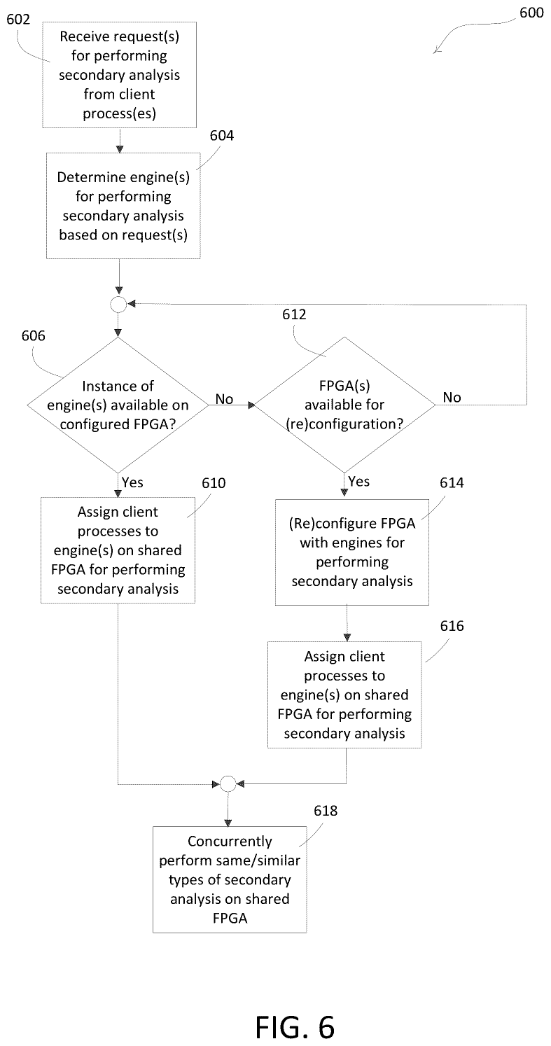

FIG. 6 is a flowchart depicting another example procedure 600 for performing secondary analysis and processing requests from client processes. The one or more portions of the procedure 600 may be performed by one or more computing devices. For example, the one or more portions of the procedure 600 may be performed by one or more server devices, sequencer devices, and/or client devices. One or more portions of the procedure 600 may be stored in memory as computer-readable or machine-readable instructions that may be executed by a processor of the one or more computing devices. One or more portions of the procedure 600 may be performed by one or more subsystems operating on a client device and/or a server device. For example, one or more portions of the procedure may be performed by a client process operating on a client subsystem, a bioinformatics subsystem, and/or a scheduler subsystem, as described herein.

The procedure 600 may begin at 602. As shown in FIG. 6, at 602 the scheduler subsystem may receive one or more requests from one or more client processes. The requests may be received individually or concurrently for being processed by the bioinformatics subsystem. The requests may include a request for an engine, or set of engines, installed on an FPGA for performing secondary analysis of sequencing data. There may be multiple FPGAs or FPGA boards on the computing system having an engine, or set of engines, for being assigned to a corresponding client process for performance of the requested hardware acceleration of secondary analysis. The request may include a type of secondary analysis to be performed. The request may include an identifier of an engine, or set of engines, to perform a type of secondary analysis. The request may include a version identifier of a version of software and/or engine to perform the secondary analysis.

At 604, the scheduler subsystem may determine an engine, or set of engines, for performing the requested hardware acceleration of secondary analysis. The scheduler subsystem may determine, at 606, whether there is an FPGA that is available that is configured with the engine, or set of engines, being requested. If an FPGA is available with the engines, or set of engines, for performing the secondary analysis for the client process, the scheduler subsystem may assign the client process to the engine, or set of engines, for performing secondary analysis at 610. The assignment may be performed by instructing the daemon subsystem to perform the assignment and/or establish a connection between the client process and the engine, or set of engines, on the FPGA for servicing the requests.

The FPGA may have multiple instances of the same engine, or set of engines, thereon, such that multiple client processes may share the FPGA resources by being assigned to different instances of the same engine, or set of engines. Thus, the FPGA may be a shared FPGA for performing secondary analysis (e.g., the same type of secondary analysis) for different client processes. The engines on the FPGA may be dedicated engines and/or shared engines. For example, each client process may be assigned to one or more dedicated engines and/or one or more shared engines for performing secondary analysis. A shared engine may be shared by multiple client processes for performing the same type of secondary analysis. The FPGA may have a single instance or multiple instances of a shared engine thereon.

If, at 606, the scheduler subsystem determines that an FPGA with the proper configuration is unavailable, the scheduler subsystem may determine at 612 whether there are available FPGAs for being configured/reconfigured for servicing the requests of the client processor. If the FPGAs are determined to be unavailable for configuration/reconfiguration, the scheduler subsystem may cause the client process to continue to wait for an FPGA with the proper configuration for being assigned. If an FPGA is available for configuration/reconfiguration at 612, the scheduler subsystem may instruct the daemon process operating at the bioinformatics subsystem to configure/reconfigure the FPGA. The configuration/reconfiguration may be performed at 614 by loading a partial bitstream image to the FPGA for configuring one or more engines on the FPGA. The partial bitstream image may include multiple instances of the same engine, or set of engines, (e.g., unzip engine, mapping engine, read alignment engine, sorting engine, dedup engine, zipping engine, haplotype assembly engine, haplotype alignment engine, read probability engine, etc.) configured to perform the same or similar type of secondary analysis (e.g., mapping/alignment, sorting, variant calling, etc.). Each engine may occupy a different logical portion of the FPGA.

After the FPGA has been configured/reconfigured at 614, the scheduler subsystem may assign the client process to the engine, or set of engines, on the FPGA for performing secondary analysis at 616. The assignment may be performed by instructing the daemon subsystem to perform the assignment and/or establish a connection between the client process and the engine, or set of engines, on the FPGA for servicing the requests. The FPGA may be assigned as a shared FPGA for performing secondary analysis in response to requests from multiple client process assigned to shared resources on the FPGA.

At 618, the FPGA may be implemented to concurrently perform the same or similar types of secondary analysis for multiple client processes using one or more engines on a shared FPGA. For example, the same or similar type of secondary analysis (e.g., mapping/alignment, sorting, variant calling, etc.) may be performed on different logical portions of the FPGA by each client process being assigned to a separate instance of the same engine, or set of engines, (e.g., unzip engine, mapping engine, read alignment engine, sorting engine, dedup engine, zipping engine, haplotype assembly engine, haplotype alignment engine, read probability engine, etc.) for performing the same type or similar type of secondary analysis. Each client process may also, or alternatively, be assigned to one or more shared engines for concurrently performing the same or similar type of secondary analysis. As secondary analysis is completed for each client process, the FPGAs may be reconfigured/reassigned to subsequent client processes for performing secondary analysis, as described herein.

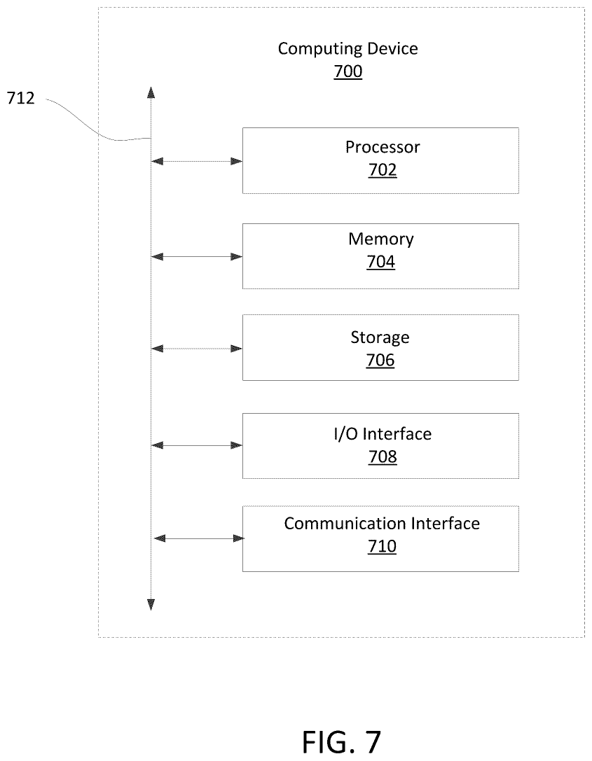

FIG. 7 is a block diagram illustrating an example computing device 700. One or more computing devices such as the computing device 700 may implement one or more features for monitoring and/or changing a version of a sequencing system to service requests from a sequencing application as described herein. For example, the computing device 700 may comprise one or more of the sequencing device 114, the client device(s) 108, and/or the server device(s) 102 shown in FIG. 1A. As shown by FIG. 7, the computing device 700 may comprise a processor 702, a memory 704, a storage device 706, an I/O interface 708, and a communication interface 710, which may be communicatively coupled by way of a communication infrastructure 712. The computing device 700 may include fewer or more components than those shown in FIG. 7.

The processor 702 may include hardware for executing instructions, such as those making up a computer application or system. In examples, to execute instructions for operating as described herein, the processor 702 may retrieve (or fetch) the instructions from an internal register, an internal cache, the memory 704, or the storage device 706 and decode and execute the instructions. The memory 704 may be a volatile or non-volatile memory used for storing data, metadata, computer-readable or machine-readable instructions, and/or programs for execution by the processor(s) for operating as described herein. For example, the memory may include computer-readable or machine readable instructions that may be executed by the processor 702 to configure, assign, and/or utilize FPGAs and/or FPGA resources, as described herein. The storage device 706 may include storage, such as a hard disk, flash disk drive, or other digital storage device, for storing data or instructions for performing the methods described herein.

The I/O interface 708 may allow a user to provide input to, receive output from, and/or otherwise transfer data to and receive data from the computing device 700. The I/O interface 708 may include a mouse, a keypad or a keyboard, a touch screen, a camera, an optical scanner, network interface, modem, other known I/O devices or a combination of such I/O interfaces. The I/O interface 708 may include one or more devices for presenting output to a user, including, but not limited to, a graphics engine, a display (e.g., a display screen), one or more output drivers (e.g., display drivers), one or more audio speakers, and one or more audio drivers. The I/O interface 708 may be configured to provide graphical data to a display for presentation to a user. The graphical data may be representative of one or more graphical user interfaces and/or any other graphical content.

The communication interface 710 may include hardware, software, or both. In any event, the communication interface 710 may provide one or more interfaces for communication (such as, for example, packet-based communication) between the computing device 700 and one or more other computing devices and/or networks. The communication may be a wired or wireless communication. As an example, and not by way of limitation, the communication interface 710 may include a network interface controller (NIC) or network adapter for communicating with an Ethernet or other wire-based network or a wireless NIC (WNIC) or wireless adapter for communicating with a wireless network, such as a WI-FI.

Additionally, the communication interface 710 may facilitate communications with various types of wired or wireless networks. The communication interface 710 may also facilitate communications using various communication protocols. The communication infrastructure 712 may also include hardware, software, or both that couples components of the computing device 700 to each other. For example, the communication interface 710 may use one or more networks and/or protocols to enable a plurality of computing devices connected by a particular infrastructure to communicate with each other to perform one or more aspects of the processes described herein. To illustrate, the sequencing process may allow a plurality of devices (e.g., a client device, sequencing device, and server device(s)) to exchange information such as sequencing data and error notifications.

In addition to what has been described herein, the methods and systems may also be implemented in a computer program(s), software, or firmware incorporated in one or more computer-readable media for execution by a computer(s) or processor(s), for example. Examples of computer-readable media include electronic signals (transmitted over wired or wireless connections) and tangible/non-transitory computer-readable storage media. Examples of tangible/non-transitory computer-readable storage media include, but are not limited to, a read only memory (ROM), a random-access memory (RAM), removable disks, and optical media such as CD-ROM disks, and digital versatile disks (DVDs).

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure.

What is claimed is:

1. A computer-implemented method capable of leveraging hardware acceleration for performing secondary analysis utilizing a plurality of field programmable gate arrays (FPGAs) installed on at least one device, the method comprising:
receiving, at a scheduler subsystem operating on the at least one device, a plurality of requests for performing hardware acceleration of secondary analysis of sequencing data from a plurality of client processes;
in response to at least one request of the plurality of requests, configuring at least one FPGA with multiple instances of an engine, or set of engines, configured to perform a same type of secondary analysis, wherein a first instance of the engine, or set of engines, is configured to perform the same type of secondary analysis as a second instance of the engine, or set of engines, wherein the engine, or set of engines, of the first instance resides in a different logical portion of the at least one FPGA than the engine, or set of engines, of the second instance;
assigning, by the scheduler subsystem, a first client process of the plurality of client processes to the first instance and a second client process of the plurality of client processes to the second instance to perform the same type of secondary analysis for the first client process and the second client process; and
concurrently performing the same type of secondary analysis on the first instance and the second instance of the engine, or set of engines, on the at least one FPGA.

2. The computer-implemented method of claim 1, wherein the engine, or set of engines, of the first instance is a dedicated engine, or set of engines, for the first client process, and wherein the engine, or set of engines, of the first instance is a dedicated engine, or set of engines, for the second client process.

3. The computer-implemented method of claim 2, further comprising:
assigning, by the scheduler subsystem, the first client process and the second client process to a shared engine, or set of engines, configured to perform a type of secondary analysis on the at least one FPGA; and
concurrently performing the type of secondary analysis on the shared engine for the first client process and the second client process, wherein the secondary analysis is performed on the shared engine by time-slicing tasks to be performed on the shared engine for the first client process and the second client process.

4. The computer-implemented method of claim 1, wherein the at least one FPGA is configured with the multiple instances of the engine, or set of engines, using a same bitstream image.

5. The computer-implemented method of claim 1, wherein a first FPGA of the plurality of FPGAs is configured to map or align the sequencing data, and wherein a second FPGA of the plurality of FPGAs is configured to perform variant calling on the sequencing data, and wherein the at least one FPGA comprises the first FPGA or the second FPGA.

6. The computer-implemented method of claim 1, wherein the engine, or set of engines, comprise at least one of an unzip engine configured to decompress a received file comprising the sequencing data, a zip engine configured to compress the sequencing data, a mapping engine configured to map or align the sequencing data, or a variant calling engine configured to predict variant calls based on the sequencing data.

7. The computer-implemented method of claim 1, wherein the plurality of FPGAs comprises 2 FPGAs or 4 FPGAs.

8. A system capable of leveraging hardware acceleration for performing secondary analysis, the system comprising:
a plurality of field programmable gate arrays (FPGAs); and
at least one processor configured to:
receive a plurality of requests for performing hardware acceleration of secondary analysis of sequencing data from a plurality of client processes;
in response to at least one request of the plurality of requests, configure at least one FPGA with multiple instances of an engine, or set of engines, configured to perform a same type of secondary analysis, wherein a first instance of the engine, or set of engines, is configured to perform the same type of secondary analysis as a second instance of the engine, or set of engines, wherein the engine, or set of engines, of the first instance resides in a different logical portion of the at least one FPGA than the engine, or set of engines, of the second instance;
assign a first client process of the plurality of client processes to the first instance and a second client process of the plurality of client processes to the second instance to perform the same type of secondary analysis for the first client process and the second client process; and
wherein the at last one FPGA is configured to concurrently perform the same type of secondary analysis on the first instance and the second instance of the engine, or set of engines.

9. The system of claim 8, wherein the engine, or set of engines, of the first instance is a dedicated engine, or set of engines, for the first client process, and wherein the engine, or set of engines, of the first instance is a dedicated engine, or set of engines, for the second client process.

10. The system of claim 9, wherein the at least one processor is configured to:
assign the first client process and the second client process to a shared engine, or set of engines, configured to perform a type of secondary analysis on the at least one FPGA; and
wherein the at least one FPGA is configured to concurrently perform the type of secondary analysis on the shared engine for the first client process and the second client process, wherein the at least one FPGA is configured to perform the secondary analysis on the shared engine by time-slicing tasks to be performed on the shared engine for the first client process and the second client process.

11. The system of claim 8, wherein the at least one FPGA is configured with the multiple instances of the engine, or set of engines, using a same bitstream image.

12. The system of claim 8, wherein a first FPGA of the plurality of FPGAs is configured to map or align the sequencing data, and wherein a second FPGA of the plurality of FPGAs is configured to perform variant calling on the sequencing data, and wherein the at least one FPGA comprises the first FPGA or the second FPGA.

13. The system of claim 8, wherein the engine, or set of engines, comprise at least one of an unzip engine configured to decompress a received file comprising the sequencing data, a zip engine configured to compress the sequencing data, a mapping engine configured to map or align the sequencing data, or a variant calling engine configured to predict variant calls based on the sequencing data.

14. The system of claim 8, wherein the plurality of FPGAs comprises 2 FPGAs or 4 FPGAs.

15. At least one computer-readable medium having stored thereon instructions that are configured to, when executed by at least one processor, cause the at least one processor to:
receive a plurality of requests for performing hardware acceleration of secondary analysis of sequencing data from a plurality of client processes;
in response to at least one request of the plurality of requests, configure at least one FPGA with multiple instances of an engine, or set of engines, configured to perform a same type of secondary analysis, wherein a first instance of the engine, or set of engines, is configured to perform the same type of secondary analysis as a second instance of the engine, or set of engines, wherein the engine, or set of engines, of the first instance resides in a different logical portion of the at least one FPGA than the engine, or set of engines, of the second instance;
assign a first client process of the plurality of client processes to the first instance and a second client process of the plurality of client processes to the second instance to perform the same type of secondary analysis for the first client process and the second client process, wherein the at last one FPGA is configured to concurrently perform the same type of secondary analysis on the first instance and the second instance of the engine, or set of engines.

16. The at least one computer-readable medium of claim 15, wherein the engine, or set of engines, of the first instance is a dedicated engine, or set of engines, for the first client process, and wherein the engine, or set of engines, of the first instance is a dedicated engine, or set of engines, for the second client process.

17. The at least one computer-readable medium of claim 15, wherein the instructions are configured to cause the at least one processor to:
assign the first client process and the second client process to a shared engine, or set of engines, configured to perform a type of secondary analysis on the at least one FPGA; and
wherein the at least one FPGA is configured to concurrently perform the type of secondary analysis on the shared engine for the first client process and the second client process, wherein the at least one FPGA is configured to perform the secondary analysis on the shared engine by time-slicing tasks to be performed on the shared engine for the first client process and the second client process.

18. The at least one computer-readable medium of claim 15, wherein the at least one FPGA is configured with the multiple instances of the engine, or set of engines, using a same bitstream image.

19. The at least one computer-readable medium of claim 15, wherein a first FPGA of the plurality of FPGAs is configured to map or align the sequencing data, and wherein a second FPGA of the plurality of FPGAs is configured to perform variant calling on the sequencing data, and wherein the at least one FPGA comprises the first FPGA or the second FPGA.

20. The at least one computer-readable medium of claim 15, wherein the engine, or set of engines, comprise at least one of an unzip engine configured to decompress a received file comprising the sequencing data, a zip engine configured to compress the sequencing data, a mapping engine configured to map or align the sequencing data, or a variant calling engine configured to predict variant calls based on the sequencing data.

21-43. (canceled)
